(19) United States
(12) Patent Application Publication
(10) Pub. No.: US 2025/0271528 A1
(43) Pub. Date: Aug. 28, 2025

(54) SYSTEM AND METHOD FOR MAGNETIC RESONANCE IMAGING USING LARGE LANGUAGE MODEL AGENTS

(71) Applicant: Case Western Reserve University, Cleveland, OH (US)

(72) Inventors: Anuj Sharma, Cleveland, OH (US); William Grissom, Cleveland, OH (US); Mark Griswold, Cleveland, OH (US); Dan Ma, Cleveland, OH (US); Andrew Dupuis, Cleveland, OH (US)

(21) Appl. No.: 19/064,621

(22) Filed: Feb. 26, 2025

Related U.S. Application Data
(60) Provisional application No. 63/557,916, filed on Feb. 26, 2024.

(51) Int. Cl. G01R 33/56; G01R 33/48; G06T 11/00; G16H 10/60; G16H 30/40

(52) U.S. Cl. CPC G01R 33/5608; G01R 33/4828; G06T 11/003; G16H 10/60; G16H 30/40; G06T 2210/41

(57) ABSTRACT

A system for automating a magnetic resonance imaging (MRI) pipeline for a patient is provided. The system includes one or more processors that are configured to receive information that includes patient data and access one or more trained LLMs. The one or more processors are further configured to apply the patient data to the one or more trained LLMs to generate an MRI protocol that includes one or more pulse sequences and pulse sequence parameters. The one or more processors are further configured to store the MRI protocol.

### CROSS-REFERENCE TO RELATED APPLICATIONS

This application claims priority under 35 U.S.C. § 119 to U.S. Provisional Patent Application No. 63/557,916 filed on Feb. 26, 20204. The entire contents of which is incorporated herein by reference.

### STATEMENT REGARDING FEDERALLY SPONSORED RESEARCH

This invention was made with government support under EB032709 awarded by the National Institutes of Health. The government has certain rights in the invention.

### BACKGROUND

Magnetic resonance imaging (MRI) can be performed with a large variety of pulse sequences, such as gradient echo, spin echo, fast spin echo, echo-planar imaging, fast low angle shot, fluid-attenuated inversion recovery, steady state free precession, diffusion-weighted imaging, and so forth. Even more, each sequence type has a variety of parameters, such as field of view (FOV) size, matrix size, flip angle, echo time, repetition time, inversion time, partial Fourier level, k-space sampling pattern, slice thickness, slice orientation, imaging extent, fat saturation switch, diffusion weighting, and so forth, that can be tuned to provide varying contrasts and resolutions. Such imaging sequences and parameters need to be chosen carefully in order to provide high-quality images that provide the right type of clinically relevant information. Thus, pulse sequence selection and protocol generation informed by the specific clinical questions underlying the study is a complex process that is subject to wide variability created by doctors and imaging technicians, which can undermine image quality, clinical utility, and/or longitudinal evaluation.

### SUMMARY OF THE DISCLOSURE

The present disclosure addresses the aforementioned drawbacks by providing a system and method for automating a magnetic resonance imaging (MRI) pipeline for a patient.

In some aspects, a system for automating an MRI pipeline for a patient is provided. The system includes one or more processors that are configured to receive information that includes patient data and access one or more trained large language models (LLMs). The one or more processors are further configured to apply the information to the one or more trained LLMs to generate an MRI protocol including one or more pulse sequences with pulse sequence parameters. The system also includes an MRI system that is configured to acquire imaging data from the patient according to the MRI protocol.

In other aspects, a method for automating generation of a magnetic resonance imaging protocol for a patient is provided. The method includes using one or more processors to receive information that includes patient data, accessing one or more trained LLMs, and applying the patient data to the one or more trained LLMs to generate an MRI protocol that includes one or more pulse sequences with pulse sequence parameters. The method also includes storing the MRI protocol.

In still other aspects, a system for automating an MRI pipeline for a patient is provided. The system includes one or more processors that are configured to receive information that includes patient data and access one or more trained LLMs. The one or more processors are further configured to apply the information to the one or more trained LLMs to generate an MRI protocol that includes one or more pulse sequences with pulse sequence parameters. The one or more processors are further configured to store the MRI protocol.

These are but a few, non-limiting examples of aspects of the present disclosures. Other features, aspects and implementation details will be described hereinafter.

### BRIEF DESCRIPTION OF THE DRAWINGS

Various objects, features, and advantages of the disclosed subject matter can be more fully appreciated with reference to the following detailed description of the disclosed subject matter when considered in connection with the following drawings, in which like reference numerals identify like elements.

FIG. 1A shows a schematic of an example system that can automate an imaging pipeline.

FIG. 1B shows a schematic of another example system that can automate an imaging pipeline.

FIG. 2 shows a flowchart setting for the steps of an example process that can use a large language model or other machine learning model to automate an imaging pipeline.

FIG. 3 shows a flowchart setting for the steps of an example process that can be used to train one or more large language models or other machine learning models to automate an imaging pipeline.

FIG. 4A shows an example radiofrequency pulse generated by a large language model in accordance with the present disclosure.

FIG. 4B shows an example slice profile simulated by a large language model in accordance with the present disclosure.

FIG. 5 shows a schematic representation of an example LLM multi-agent system used for magnetic resonance exam optimization in accordance with the present disclosure.

FIG. 6 provides a table of sequence generation tools and parameters available to the Sequence Agent in an example implementation of the methods described herein.

FIG. 7 provides examples of a multi-agent system's outputs generated for three example clinical cases.

FIG. 8 shows an example response to a prompt generated by an LLM in accordance with the present disclosure.

FIG. 9 shows example images acquired using sequences generated by an example multi-agent system.

FIG. 10 is a block diagram of an example magnetic resonance imaging (“MRI”) system that can implement the methods described in the present disclosure.

FIG. 11 is a block diagram of an example imaging automation system that can implement the methods of the present disclosure.

FIG. 12 is a block diagram of example components that can implement the system of FIG. 11.

### DETAILED DESCRIPTION

Before any aspects of the present disclosure are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

Magnetic Resonance Imaging (MRI) is a medical imaging modality that can create detailed anatomical, functional, and quantitative images of the human body. During an MRI scan, a pulse sequence is played out to generate, spatially encode, and receive tissue-dependent radio-frequency signals. A typical pulse sequence comprises radiofrequency (RF) pulses, gradient waveforms and analog-to-digital converter (ADC) events. The order, timing and properties of these events are controlled by a computer program. During a scan, the computer program is loaded onto the scanner system, which may be referred to as a spectrometer, in the form of an event table. The spectrometer sends temporally ordered instructions to the scanner hardware according to the event table. The RF pulses and gradient waveforms are transmitted using electrical coils surrounding or positioned near the imaging volume. The generated signal is received using RF coils and digitized using ADC circuitry. Finally, the digital signals are processed using a computer program to reconstruct images that are post-processed and displayed to the operator. The reconstruction pipeline also often includes several tunable parameters and variations (e.g., order of operations) that can affect image quality.

One of the advantages of MRI is that it can provide a large variety of image contrasts for the same underlying anatomical or physiological state. The images can be made sensitive to different contrast mechanisms by simply modifying the pulse sequence type and parameters. Many pulse sequences exist, including gradient echo (GRE), fast low angle shot (FLASH), spin echo (SE), fast spin echo (FSE), echo-planar imaging (EPI), fluid-attenuated inversion recovery (FLAIR), steady state free precession (SSFP), and so forth. These pulse sequences can be further tuned by setting sequence parameters, such as flip angle (FA), repetition time (TR), echo time (TE), inversion time (TI), partial Fourier level, receiver bandwidth, and so forth, that modify contrast levels, such as T₁-weighting, T₂-weighting, T₂*-weighting, proton density weighting, and so on. Moreover, these sequences can be combined with other preparatory sequences or temporal repetition that generate other contrasts to provide various types of imaging, including magnetization preparation (MP), diffusion weighted imaging (DWI), perfusion weighted imaging (PWI), diffusion tensor imaging (DTI), magnetic resonance fingerprinting (MRF), functional imaging (fMRI), arterial spin labeling (ASL), susceptibility weighted imaging (SWI), and so on. Imaging can also be performed with the presentation of exogenous contrast agents, like gadolinium (Gd)-, iron-, or manganese-based compounds, which affect relaxation times. Thus, there is a large variety of pulse sequences, imaging protocols, and reconstruction methods to choose from.

For a given patient, a small set of pulse sequences must be selected to acquire the MR data needed to answer the clinical question posed in the imaging order, while controlling or minimizing imaging time. This is not a straightforward task. In the clinical workflow, the set of pulse sequences used to image a patient is selected based on disease symptoms and inputs from the referring physician, the imaging technologist, the radiologist, and possibly also the electronic health record (EHR) system. Sometimes exploratory scans are first performed, the patient is allowed to return home and called back later for more targeted imaging sessions to further diagnose and/or evaluate disease progression. From the first physical examination of the patient to the imaging session, there are multiple healthcare personnel and computer systems involved. In a typical workflow, a referring physician provides a radiology order, which may include disease symptoms, a possible diagnosis, or other inputs, to the radiology department. Next, a radiologist prescribes the MRI scans by determining the appropriate protocol, which is then carried out by an MRI technologist who sets many of the protocol parameters. This multi-step process and the manual selection of pulse sequences and parameters can lead to errors due to human factors, which can cause rescanning, high recall rate, low clinical throughput, reduced image quality, inconsistent image quality and type across a patient population, and increased cost to the patient and the healthcare system. The present disclosure provides systems and methods that can automate some or all steps of this complex workflow.

For some anatomical regions, there are well established guidelines. For example, the American College of Radiology has published the Prostate Imaging Reporting and Data System (PI-RADS) and Breast Imaging Reporting and Data System (BI-RADS) for structured reporting of image findings related to prostate and breast cancer, respectively. However, such guidelines are not widely available for all anatomies and disease states, and the entire suite of scans in these protocols may not be relevant to all cases. Thus, the selection of pulse sequences can vary widely since it is largely based on the collective past clinical experience and knowledge of the involved healthcare personnel.

To address the above challenges, the present disclosure describes systems and methods to prescribe customized MRI pulse sequences based on information that may include the patient data, which may include a patient's health record, and the healthcare provider's imaging orders or preferences. As will be described, the systems and methods can employ an interconnected system of large language model (LLM) agents. The LLM agents can be configured to prescribe and optionally optimize MRI pulse sequences. The MRI pulse sequence may be customized to the patient, such as based on the patient's health records, and the imaging order. For example, the trained LLM can analyze physician notes to determine a set of tissue contrasts to image. As another example, the trained LLM can generate a list of tissue parameter maps to measure based on the identified clinical question.

The LLM agents can further launch data reconstruction and quality analyses of the resulting images and parametric tissue maps to evaluate whether they are sufficient to answer the clinical questions. The LLM agents can also assess whether rescans or additional scans (e.g., other contrasts or imaging parameters) are needed.

In some configurations, LLMs can be used to allow a user to interact with an imaging system at various points throughout the imaging pipeline. As a non-limiting example, a user could prompt the system by saying or inputting a phrase. The phrase can be interpreted by one of the LLM agents, which can trigger various tasks of the imaging pipeline. As non-limiting examples, the input phrase may include, “design an imaging protocol to determine whether patient X has breast cancer”, “design an imaging protocol to characterize patient X's extent of prostate cancer,” “design an imaging protocol to assess patient X's neurological function,” “design a pulse sequence protocol for diffusion weighted imaging or patient X's liver tumor,” “design an imaging protocol for patient X based on their health records from the last 10 years”, “produce a reconstruction pipeline for patient X's imaging protocol,” “retrieve relevant health data for patient X,” and so forth. In some implementations, the output of an LLM agent can provide input of a second LLM agent. In this way, the LLM agents can be interconnected to provide an automated imaging pipeline. This connection can be facilitated by a so-called “Controller Agent,” as will be described further below.

LLMs are deep neural network models that are trained to perform natural language processing tasks such as language interpretation and generation. In the disclosed method, several LLM agents are created to perform the tasks within an automated pipeline, starting from interpreting the patient's electronic health record (EHR) to generating a final report on the findings from the imaging session.

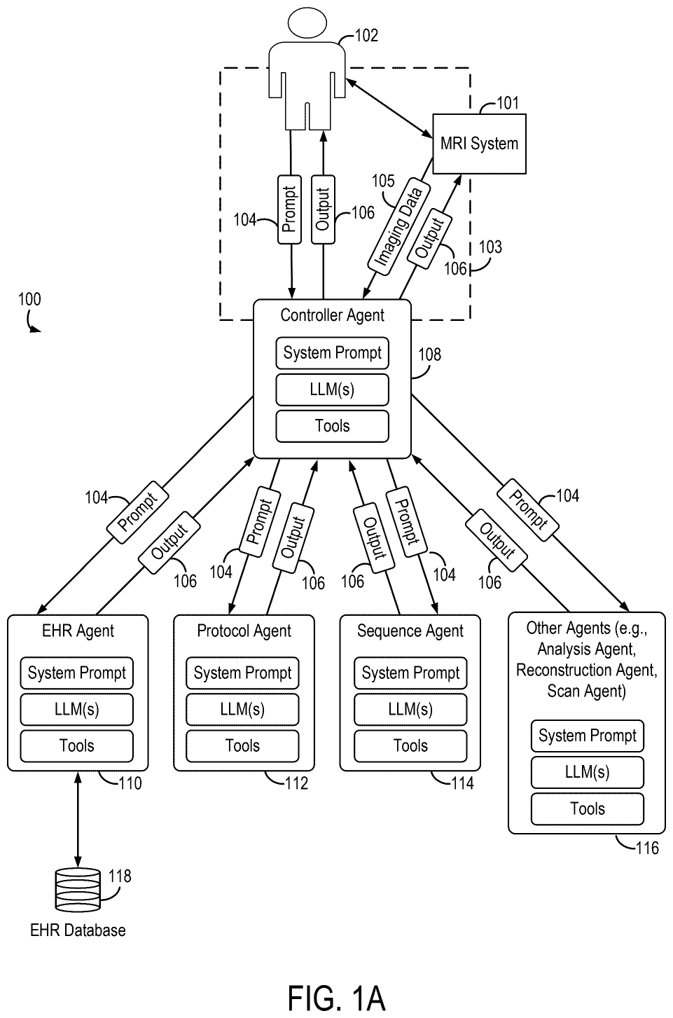

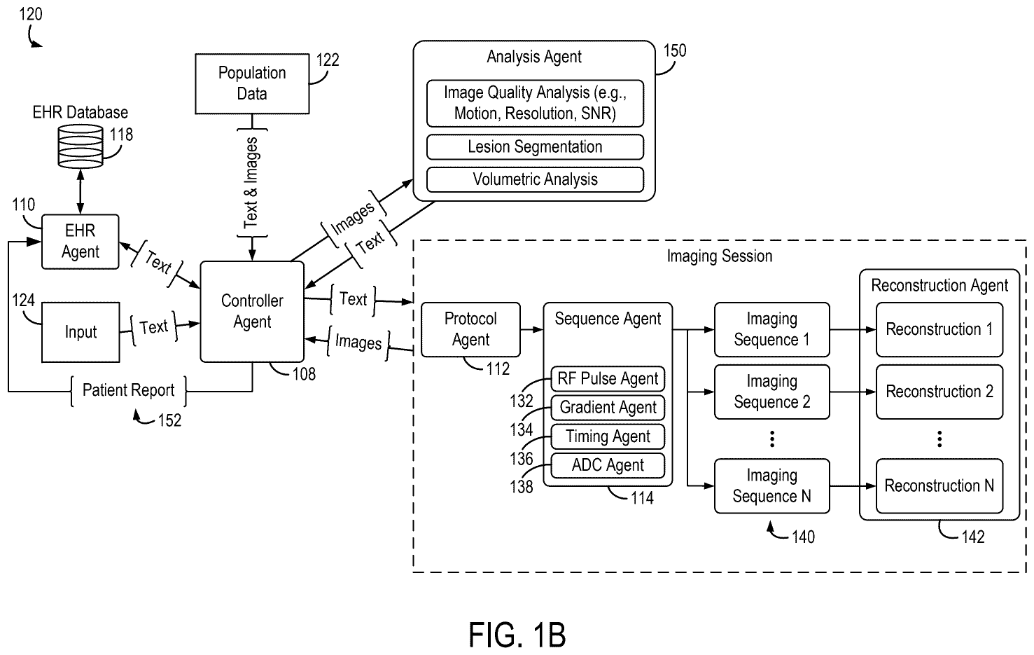

FIGS. 1A and 1B schematically show example systems 100 and 120 that may be used to coordinate and improve an MRI acquisition pipeline in an automated way. The systems may include one or more memories that can store data, such as instructions, large language models and model parameters, patient data, imaging orders, pulse sequence or reconstruction parameters, raw MRI data, reconstructed images and parameter maps, image analysis results, scanner event tables, and so forth. The system can also include one or more processors that are configured to execute stored instructions.

In some configurations, the systems 100 and 120 apply one or more LLMs that are trained to generate optimized or otherwise desirable pulse sequences that answer a particular clinical question. For example, the LLMs can generate RF pulses, gradient waveforms, and timing parameters to produce pulse sequences that fit into a constrained scan time. The clinical question can be established manually by a user or generated by an LLM based on patient or EHR data. In this way, the LLM can interpret the EHR data to produce instructions for the scanner for an imaging session.

The use of LLMs can be facilitated by a series of agents. Each agent has access to one or more LLMs. In some configurations, the LLMs may conveniently include general and pre-trained models, such as Generative Pre-trained Transformer 4 (GPT 4). These general models can be harnessed for specific tasks using system prompts set by a developer and based on access to relevant tools. In other configurations, the LLMs can be specifically trained for given tasks (e.g., trained only on radiological data). In other configurations, the LLMs can be based on general models (e.g., GPT 4) and fine-tuned for a specific application (e.g., interpreting EHR data, generating RF pulses).

Referring to FIG. 1A, in some configurations, the system 100 can be configured to receive information from a user 102 or a system (e.g., a clinical software system). The information may include patient data for a patient or group of patients or other data. For example, the patient data may include a patient identification number, a radiology order, previously acquired images, physician notes, or a clinical question of interest. The information may also include institutional standards, radiologist preferences, or other relevant information (e.g., the MRI system model, MRI coils available, maximum allowed scan time. In some configurations, the system 100 can apply such information as a user prompt 104 to one or more trained LLMs. This prompt 104 can be provided as a text prompt, as another type of data (e.g., patient identification number, physician notes, patient demographic data, images, imaging data, MRI system identification, hardware limits, and so forth), or a combination thereof.

The prompt 104 can be provided manually by a user 102, such as a referring physician, a radiologist, a nurse, an MRI technologist, or another clinician. The prompt 104 can also be provided automatically by another computer system (e.g., automatically accessing after visit notes from a clinical software). The one or more trained LLMs generate an MRI protocol, including pulse sequences and pulse sequence parameters based on the information provided. Once the MRI protocol is generated, it can be stored by the system 100 or communicated back to the user 102 as an output 106.

In some configurations, the system 100 can communicate directly or indirectly with an MRI system 101. As one example, the output 106 may include scanner instructions that control the MRI system 101 to acquire imaging data or MRI data, according to the protocol designed by the system 100. Once the MRI system 101 executes the imaging protocol, the imaging data 105 may be returned to the system 100. The imaging data 105 may include raw k-space data, filtered k-space data, reconstructed images, protocol information (e.g., pulse sequence parameters used), other data provided by the MRI system 101, or some combination thereof.

In this way, the system 100 can communicate with a user 102 via a user interface or an MRI system 101 or both using a communication system 103. Such communication system 103 provides a connection between a user 102, the system 100, and an MRI system 101, or some combination thereof.

The system 100 may include one or more processors that are configured to implement a variety of agents that are interconnected via natural language prompts. One of the agents is a user-facing agent, which may be referred to as the ‘Controller Agent’ 108 that oversees and coordinates the actions of other agents. In this way, the Controller Agent 108 takes a prompt 104 from a user 102 or clinical system. The Controller Agent 108 can then control each of the other agents. In some implementations, the Controller Agent 108 can be configured to generate and provide a prompt 104 to each of the other agents. The prompt 104 can include text prompts generated by the Controller Agent 108. The prompt 104 may also include other relevant data, such as a patient MRN, demographic data, imaging data, MRI system data, scan constraints (e.g., time, hardware), and so forth. These prompts can be provided to various agents, which may include an ‘EHR Agent’ 110, a ‘Protocol Agent’ 112, a ‘Sequence Agent’, 114, and other agents 116 (e.g., ‘Analysis Agent’, ‘Reconstruction Agent’, ‘Scan Agent’, and so forth), for example.

Each agent is task-specific and may be configured to function independently from the other agents. Each agent has a responsibility that can be configured by a developer. For example, the agents can be configured with agent-specific system prompts that prepare each agent to carry out its responsibility (e.g., “You are an expert MR pulse sequence programming assistant who chooses optimal pulse sequence parameters and writes pulse sequences,” provided to the Sequence Agent 114). The agents are configured to use one or more LLMs to perform their given tasks. They can also be configured to have access to various tools (e.g., external computer programs, physics-based tools) that can be used to accomplish the tasks.

Thus, each agent can be provided with a prompt 104 from the Controller Agent 108 at runtime. In this way, the prompts 104 can dynamically change on a patient-specific basis and throughout the process of generating the imaging protocol. The agents can apply each prompt to an LLM to produce an output 106 that is communicated back to the Controller Agent 108. The Controller Agent 108 facilitates completion of the main task by selectively calling upon various agents with appropriate prompts 104. The Controller Agent 108 can also iteratively call the agents if needed. The Controller Agent 108 then provides an output 106 to the user 102 or to control the actions of an MRI 101 or other clinical system.

One or more processors can be configured to execute a so-called ‘EHR Agent.’ The EHR Agent is configured to retrieve electronic health records (EHR) from a hospital computer system or EHR database 118. Such EHR data can include historical records of the patient's health or demographics, current and previous symptoms, prior clinical diagnoses, clinician notes and reports, potential diagnoses to investigate, diagnosis questions, clinical question of interest, and so forth. In some implementations, the agent can be configured to apply the EHR or patient data to a trained LLM that analyzes the patient data to determine the clinically relevant patient data. For example, the EHR Agent may identify recent potential diagnoses and related historical patient data (e.g., previously acquired images). The EHR Agent may determine what patient data is clinically relevant and generate a clinical goal or clinical question of interest (e.g., evaluate patient for dementia). In other configurations, the clinical question(s) may be provided manually by a clinician or other user 102. The clinical goal or other relevant clinical information is communicated back to the Controller Agent 108 as an output 106.

The system may also include a so-called ‘Protocol Agent’ 112 that applies the relevant clinical information to one or more LLMs trained to determine a customized MRI protocol. The MRI protocol includes one or more pulse sequence types. For example, the MRI protocol may include a desired set of tissue parameters, quantitative parameters maps, or contrast types to be measured. In this way, the Protocol Agent 112 can determine a customized MRI protocol that is well-suited to answer the posed clinical question. As a non-limiting example, based on a potential diagnosis of breast cancer, the LLM may prescribe a DWI pulse sequence, a pre-contrast pulse sequence, and a dynamic contrast-enhanced protocol (e.g., seven timepoints throughout the course of contrast uptake).

The corresponding pulse sequence parameters are assigned by the so-called ‘Sequence Agent’ 114. The Sequence Agent 114 uses an LLM to determine pulse sequence parameters for each sequence of the protocol. Such pulse sequence parameters may include flip angle, pulse sequence timing, types or details of preparatory pulses (e.g., diffusion weights), spatial or temporal resolution, imaging extent, slice geometry (e.g., positions and orientations, 2D vs. 3D), and so forth. As a non-limiting example, the LLM may generate an MR Fingerprinting (MRF) pulse sequence with parameters (e.g., TR, TE and flip angle) that make the sequence more sensitive to the range of tissue parameter values (e.g., T₁, T₂) expected from the patient's clinical indication. Applying the LLM to generate an MRF pulse sequence may be particularly advantageous because the MRF sequence provides many tunable parameters that can be adjusted to increase a desired contrast in a way that is well-suited for evaluating a particular condition or disease. For example, the varying parameters that change throughout the pulse sequence can be tuned such that the LLM determined a time course for the varying pulse sequence parameters (e.g., flip angle, echo time, repetition time, and so forth).

The Sequence Agent 114 can provide an output 106 as instructions for an MRI system or user 102 who controls an MRI system (e.g., technician). These scan instructions may be provided as a list of sequence parameters corresponding to each sequence type. In other configurations, the instructions can be provided as an MRI sequence file that can be provided directly to the MRI system 101 to control the MRI system 101 to execute the prescribed sequence to acquire raw data. The MRI sequence file may store an event table that includes timing parameters and a series of event blocks, such as radiofrequency (RF) pulse blocks and gradient waveform blocks. In this way, the event table defines the switching of the RF pulses and gradient fields of the MRI system.

Thus, the Sequence Agent 114 may be given access to physics-based tools that design sequences given a set of sequence parameters or other constraints provided by the agent. For example, the tools may include a program that designs RF pulses, gradient waveforms, other pulse sequence blocks, or full sequences (e.g., CreateGRESequence( ), CreateTSESequence( ), and so forth) using various physics-based methods. As a non-limiting example, the LLM can access a tool that generates an RF waveform given a desired sequence type and slice profile or other constraints. An LLM may be used to verify that the pulse sequence parameters generated fall within hardware and patient safety limits. For example, the LLM can access a tool that calculates hardware requirements (maximum slew rate, maximum gradient strength, specific absorption rate (SAR), and so on) and compares such requirements to hardware and safety limits. The process can be iteratively repeated to provide high quality imaging protocols while maintaining hardware and safety limits.

As a non-limiting example, the Sequence Agent 114 can then take a prompt 104 (e.g., “generate a high-resolution T₁-weighted sequence for full brain imaging that takes less than 3 minutes to acquire”), which can be provided by the Controller Agent 108 at runtime. The task can be carried out by applying the prompt 104 to one or more LLMs. After the agent has completed its task, it can provide the appropriate output 106 back to the Controller Agent 108, which can prompt other agents to carry out their tasks as needed based on the output 106.

A processor is configured to reconstruct the raw data. In some implementations, a trained LLM can be used to generate a reconstruction pipeline that can be used to produce images (e.g., anatomical images, 4D images, functional images) or parameter maps (T₁ map, fat fraction map, apparent diffusion coefficient map, etc.). In other implementations, a trained neural network can be used to execute image reconstruction from the imaging data, which may include raw k-space data received from the MRI system 101.

In some implementations, the LLM can generate a reconstruction pipeline, determine reconstruction parameters, or execute image reconstruction based on the clinical question, protocol parameters, other EHR data, or a combination thereof. For example, the LLM can select a series of computer program modules that each performs a specific image reconstruction or processing step. As a non-limiting example, individual modules may be programmed to perform tasks including parallel imaging, partial Fourier reconstruction, Fourier transforms, motion correction, distortion correction, filtering, noise reduction, and so forth. A module may also be configured to perform MRF tissue parameter map generation using dictionary-based or other MRF analysis techniques. The LLM can select the modules, module parameters (e.g., level of noise reduction, type of parallel imaging reconstruction), and order of reconstruction tasks. In some implementations, the image reconstruction can also include generation of parameter maps (e.g., T₁, T₂, T_{2*}, apparent diffusion coefficient, blood flow speed, fat fraction, likelihood of cancer lesion, segmentation and tissue identification, and so forth). Thus, the LLM can also assign a computer module that calculates tissue parameters based on the imaging data to produce parameter maps using a physics-based method (e.g., linear regression fit) or trained machine learning algorithm. The processor can store the raw data, reconstructed images, parameter maps, or combination thereof in a memory for future use.

The processor may be further configured to analyze the images or parameter maps by applying the reconstructed images to an LLM. For example an LLM may be trained to generate a report that outlines findings from the imaging session. As a non-limiting example, such a report may include description of lesion size, contrast uptake, spiculation, and diffusion characteristics. As another non-limiting example, the report may include a risk score for the disease or condition of interest, provide a diagnosis estimate, or quantify severity of disease or disease progression. As another non-limiting example, the report may include details of the protocol used and resulting image quality. In some implementations the analysis LLM may be trained on a large group of radiologist reports and corresponding images. Thus, the LLM can analyze the images to generate a report similar to that produced by a radiologist in standard practice. This report can be communicated to the user 102 as an output 106 or stored for later clinical or other use.

In some implementations, the LLM can interpret the images or report to determine whether the clinical question of interest has been answered. If not, the system can recommend and execute further imaging with modified parameters or contrast methods. For example, the LLM-based system can determine that a parameter map is too blurry to provide reliable analysis of and quantification within a lesion and prompt the scanner to acquire new images with higher resolution within an imaging volume targeted to the lesion in question. In this way, the processor is configured to apply the images or report to the LLM to update the MRI protocol, the reconstruction pipeline, or both. Once the Controller Agent 108 is satisfied, the report can be communicated back to the user 102 as an output 106.

As a non-limiting example, the user 102 can provide the prompt 104 as a patient identification number (e.g., medical record number) to the Controller Agent 108. The Controller Agent 108 can enlist the EHR Agent 110 to retrieve relevant patient information from the EHR database 118. The EHR Agent can determine the appropriate clinical information desired (e.g., a clinical goal, diagnostic question) and provide the relevant information back to the Controller Agent 108 as an output 106. For example, the output 106 may be “patient previously diagnosed with multiple sclerosis.” The Controller Agent 108 can use this output to generate a prompt for the Protocol Agent 112. For example, the prompt 104, “generate a protocol to evaluate progression of multiple sclerosis.” The Protocol Agent 112 can then produce an MRI protocol as an output 106 as a list of relevant pulse sequence types, such as “brain axial T₂-weighted, brain axial T₁-weighted, and brain axial FLAIR.” Next, the Controller Agent 108 can provide the Sequence Agent 114 with a prompt 104, such as “generate a brain axial T₂-weighted sequence” or “provide sequence parameters for a brain axial T₂-weighted sequence.” The Sequence Agent 114 can provide and output 106, for example, as pulse sequence parameter or as an event table that provides instructions for an MRI system 101 to acquire data.

The system 100 may also include other agents 116. For example, a so-called ‘Reconstruction Agent’ can provide an appropriate reconstruction pipeline for the data acquired with the generated pulse sequence. A so-called ‘Analysis Agent’ can be used to analyze the images to provide feedback to the Controller Agent 108 (e.g., to prompt generation of a new protocol if the one provided did not meet the clinical goal) or to provide clinical information about the images. A Scan Agent can be used to prescribe the scan volume of interest and run the MRI system to acquire the data. In some implementations, these agents can be iteratively called by the Controller Agent 108 to improve the resulting imaging pipeline and better meet the clinical goal. For example, the process can be repeated with new parameters if the clinical question is not sufficiently answered.

The described method automates processes that can be error-prone, non-standardized, and non-repeatable when performed by humans, while acquiring images that are customized to the patient's health record and designed to answer the clinical question posed by the imaging order. The LLM agents automate the pulse sequence selection task. In addition, the LLM agents optimize the pulse sequence to the current patient, which leads to richer image information content. Furthermore, the described LLM agents can automate the EHR language interpretation and findings report. All these features of the disclosed systems and methods increase patient throughput, reduce errors due to human factors, and reduce reimaging sessions and recall rate, which reduces cost to the patient and the healthcare system.

FIG. 1B schematically shows another example implementation of a system 120. Systems 100 and 120 shown in FIGS. 1A and 1B, respectively, are provided as example configurations. It should be understood that the features of systems 100 and 120 can be combined in various combinations. In system 120, the Protocol Agent 112 and Sequence Agent 114 are configured to communicate directly such that the protocol agent 112 controls the generation of each imaging sequence 140 in the protocol. The Protocol Agent 112 can also control the reconstruction of each sequence by the reconstruction agent 142. FIG. 1B also provides additional details of the flow of data between agents and details of optional features of the systems 100 and 120.

The system 120 can be trained on population data 122. This population data 122 data can be received by the Controller Agent 108 to train or fine tune the one or more LLMs that are employed by the agents. For example, the population data 122 may include manually generated imaging protocols paired with EHR data for a group of patients. As another example, the population data 122 may include imaging protocols paired with image orders that pose clinical questions. The population data 122 may also include recall rates and recall reasons for previously performed MRI scans. The Controller Agent 108 can interpret the population data 122 to refine the imaging process.

Once trained, the Controller Agent 108 controls the imaging process. The Controller Agent 108 can receive an input 124 from a user or external system. The input 124 may include an imaging order (e.g., “MRI protocol for Alzheimer's patient”). The input 124 may also include a specific prompt (e.g., “Design an excitation pulse with 128 timepoints and time bandwidth 8”). The input 124 may alternatively include a patient identification (e.g., MRN).

Based on the input 124, the Controller Agent 108 begins to selectively call the other agents to accomplish its given task. For example, the Controller Agent 108 may begin by asking the EHR Agent 110 to securely access the hospital computer system to obtain the patient EHR based on the MRN. The EHR Agent 110 interprets the EHR data and the imaging order and recommends a set of tissue parameters (e.g., T₁, T₂, T_{2*}, proton density, and so forth) or contrasts (e.g., fat fraction, diffusion, blood flow, contrast-enhancement dynamics, and so forth) that can be measured to answer the clinical question. The Controller Agent 108 then asks the Protocol Agent 112 to develop a set of scans to measure those parameters or contrasts while minimizing scan time. In some implementations the Protocol Agent 112 is configured to develop a scanning protocol that is constrained to a maximum total imaging time.

For each scan, the Protocol Agent 112 asks the Sequence Agent 114 to fully define each scan in a manner that is optimized for the required parameter or contrast, while minimizing scan time. The Sequence Agent 114 can then invoke agents that are tasked with building each part of the imaging sequences and reconstructions. For example, the Sequence Agent 114 may employ so-called ‘RF Pulse Agent’ 132 that is responsible for building an RF pulse, ‘Gradient Agent’ 134 that defines the gradient waveforms, ‘Timing Agent’ 136 that determines the sequence timing, and ‘ADC Agent’ 138 that controls the analog to digital converter to acquire the data.

The Sequence Agent 114 can generate several imaging or pulse sequences 140 of different types as determined by the Protocol Agent 112. The Reconstruction Agent 142 can produce a reconstruction pipeline for each sequence. The reconstruction pipeline may be based on the imaging sequence 140 used, the clinical goal, feedback from the Analysis Agent 150, another input 124 from a user, or some combination thereof. The Reconstruction Agent 142 may determine the reconstruction parameters (e.g., level of denoising, types of parallel imaging reconstruction, and so forth) and order of operations (e.g., motion correction, parallel imaging reconstruction, deblurring, denoising, production of parameter maps, and so forth). Thus, the Reconstruction Agent 142 may generate a reconstruction pipeline that includes a series of computer program modules that are each configured to perform an image reconstruction step.

The scans are executed, the resulting images and parametric maps are sent back to the Controller Agent 108, which then sends them to an Analysis Agent 150 that assesses their quality and generates reports. For example, such reports may identify image quality, identify lesions, provide volumetric analyses, identify regions of interest (ROI), provide image or lesion segmentation, and so forth. In some configurations, a patient report 152 can be communicated back to the EHR Agent 110 to be stored in the EHR database 118 for future clinical use or otherwise communicated back to the user or clinician.

If the Controller Agent 108 is not satisfied that the acquired images or parameter maps answer the clinical question, it can request that the Protocol Agent 112 suggest a new scan. For example, the Protocol Agent 112 may prescribe additional scans with reduced motion sensitivity, higher SNR or spatial resolution, or additional contrast types to answer the clinical questions within the total scan time budget. As another example, the Reconstruction Agent 142 may repeat the reconstruction pipeline with new parameters to improve the image quality. At exam completion, the Controller Agent 108 produces a report that can be uploaded to the hospital computer network for consumption by the healthcare personnel.

### Neural Network Implementation

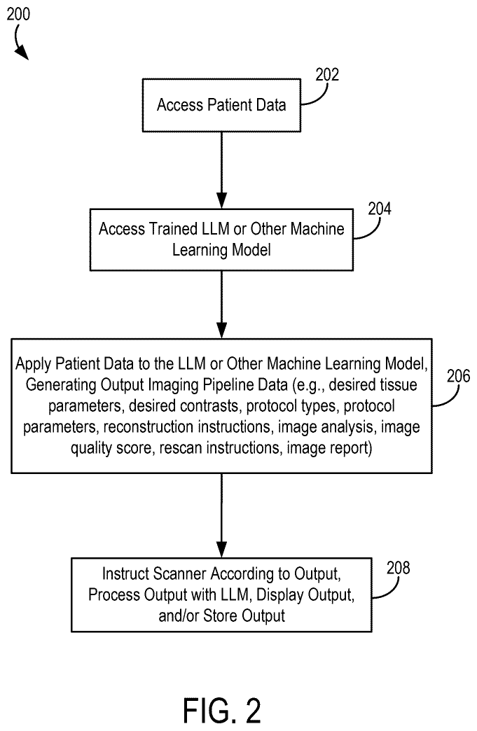

Referring now to FIG. 2, a flowchart is illustrated as setting forth the steps of an example process 200 for generating output imaging pipeline data using a suitably trained neural network, such as a large language model (LLM) or other machine learning algorithm. The systems shown in FIGS. 1A and 1B may include the use of one or more LLMs. In some implementations, each LLM is trained or fine-tuned for a particular task (e.g., identify a relevant clinical question, identify tissue parameters or contrasts to measure, generate an imaging protocol, generate a reconstruction pipeline, analyze images, generate a report, estimate diagnosis or disease progression). Thus, process 200 can be used with a variety of LLMs or other machine learning algorithms trained for various tasks at various steps of the automated pipeline.

As will be described, the neural network or other machine learning algorithm takes information (e.g., patient data, text prompt, and so forth) as input data and generates output imaging pipeline data as output data. As an example, the output imaging pipeline data can be indicative of a clinical question of interest, desired tissue parameters or contrast to be measured, a prescribed MRI sequence protocol, a reconstruction pipeline, an image analysis report (e.g., image quality, segmentation, volumetric analysis, radiologic report), a diagnosis estimate, a characterization of disease progression, or a secondary imaging protocol. As an example, the patient data may include a patient identification number, EHR data, user input (e.g., in the form of a language prompt), the patient's current or previous symptoms, clinical impressions determined by a healthcare provider, diagnosis options, open clinical questions (e.g., provided as language prompts such as “generate an imaging protocol to assess neurological function”), imaging orders, patient safety information (e.g., pacemaker or other metal implant, kidney function, circulation conditions, prescription or other drug use, pregnancy status, age and weight), MRI data (anatomical images, functional images, parameter maps), and so forth. In some implementations, the patient data may include outputs from previous use of an LLM.

### Accessing Data

The method includes accessing patient data with a computer system, as indicated at step 202. Accessing the patient data may include retrieving such data from a hospital database (e.g., EHR), memory, or other suitable data storage device or medium. Accessing the patient data may also include receiving a manually generated prompt (e.g., from a clinician). Additionally or alternatively, accessing the patient data may include acquiring imaging data with an MRI system and transferring or otherwise communicating the data to the computer system, which may be a part of the MRI system. In some implementations, accessing the patient data includes generating such patient data using the automated imaging system with earlier steps within the pipeline.

### Accessing Trained Neural Network

A trained neural network, such as an LLM, or other suitable machine learning algorithm is then accessed with the computer system, as indicated at step 204. Accessing the trained neural network may include accessing network parameters (e.g., weights, biases, or both) that have been optimized or otherwise estimated by training the neural network on training data. In some instances, retrieving the neural network can also include retrieving, constructing, or otherwise accessing the particular neural network architecture to be implemented. For instance, data pertaining to the layers in the neural network architecture (e.g., number of layers, type of layers, ordering of layers, connections between layers, hyperparameters for layers) may be retrieved, selected, constructed, or otherwise accessed.

In general, the neural network is trained, or has been trained, on training data in order to automate various steps of the imaging pipeline. An artificial neural network generally includes an input layer, one or more hidden layers (or nodes), and an output layer. Typically, the input layer includes as many nodes as inputs provided to the artificial neural network. The number (and the type) of inputs provided to the artificial neural network may vary based on the particular task for the artificial neural network.

The input layer connects to one or more hidden layers. The number of hidden layers varies and may depend on the particular task for the artificial neural network. Additionally, each hidden layer may have a different number of nodes and may be connected to the next layer differently. For example, each node of the input layer may be connected to each node of the first hidden layer. The connection between each node of the input layer and each node of the first hidden layer may be assigned a weight parameter. Additionally, each node of the neural network may also be assigned a bias value. In some configurations, each node of the first hidden layer may not be connected to each node of the second hidden layer. That is, there may be some nodes of the first hidden layer that are not connected to all of the nodes of the second hidden layer. The connections between the nodes of the first hidden layers and the second hidden layers are each assigned different weight parameters. Each node of the hidden layer is generally associated with an activation function. The activation function defines how the hidden layer is to process the input received from the input layer or from a previous input or hidden layer. These activation functions may vary and be based on the type of task associated with the artificial neural network and also on the specific type of hidden layer implemented.

Each hidden layer may perform a different function. For example, some hidden layers can be convolutional hidden layers which can, in some instances, reduce the dimensionality of the inputs. Other hidden layers can perform statistical functions such as max pooling, which may reduce a group of inputs to the maximum value; an averaging layer; batch normalization; and other such functions. In some of the hidden layers each node is connected to each node of the next hidden layer, which may be referred to then as dense layers. Some neural networks including more than, for example, three hidden layers may be considered deep neural networks.

The last hidden layer in the artificial neural network is connected to the output layer. Similar to the input layer, the output layer typically has the same number of nodes as the possible outputs. In an example in which the artificial neural network generates an image report, the output layer may include, for example, a number of different nodes, where each different node corresponds to a different feature of the report. A first node may indicate diagnosis estimate, a second node may indicate an image resolution score, a third node may indicate volume statistics of a lesion, and so on.

### Applying Data to the Neural Network

The patient data are then input to the one or more trained neural networks, generating output as output imaging pipeline data, as indicated at step 206. For example, the output imaging pipeline data may include a defined clinical question of interest, tissue parameters or contrast types of interest, a prescribed imaging protocol, reconstruction pipeline, imaging data, an imaging report, a diagnosis estimate, a risk score, and so forth, as previously described. The output imaging pipeline data may also include a recommendation for further imaging, which can prompt an LLM to generate a new imaging protocol.

### Display and/or Store Output

The output imaging pipeline data generated by inputting the patient data to the trained neural network(s) can then be used for downstream pipeline steps, displayed to a user, stored for later use or further processing, or both, as indicated at step 208. For example, the output can be used to prompt an MRI system to acquire images as prescribed by the LLM.

### Neural Network Training

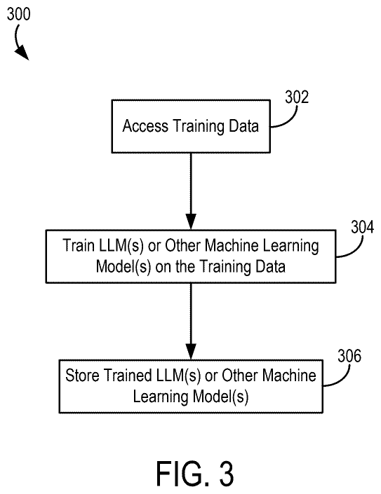

Referring now to FIG. 3, a flowchart is illustrated as setting forth the steps of an example process 300 for training one or more neural networks, such as an LLM, or other suitable machine learning algorithms on training data, such that the one or more neural networks are trained to receive patient data as input data in order to generate output imaging pipeline data as output data. The output imaging pipeline data may provide relevant clinical questions or desired measurements, MRI exam instructions, reconstruction instructions, image analysis, or image reports. Thus, process 300 can be used to train several LLMs for each desired task within the automated imaging pipeline.

In general, the neural network(s) can implement any number of different neural network architectures. For instance, the neural network(s) could implement a convolutional neural network, a residual neural network, or the like. Alternatively, the neural network(s) could be replaced with other suitable machine learning or artificial intelligence algorithms, such as those based on supervised learning, unsupervised learning, deep learning, ensemble learning, dimensionality reduction, and so on.

### Accessing Training Data

The method includes accessing training data with a computer system, as indicated at step 302. Accessing the training data may include retrieving such data from a memory or other suitable data storage device or medium. Alternatively, accessing the training data may include acquiring such data with an MRI system and transferring or otherwise communicating the data to the computer system. Alternatively, accessing the training data may include generating the training data in previous steps in the imaging pipeline (e.g., process 200) using another trained LLM or previously trained LLM. In this way, the LLM may be retrained over time based on previous uses of the LLM and associated outcomes (e.g., recall rate).

In general, the training data can include patient data from a large population. For example, such patient data may include EHR data, patient symptoms, relevant clinical questions, MRI images, clinical diagnoses, MRI protocol data, MRI image analysis data, radiological imaging reports, clinical outcomes, and so forth. Additionally, the training data may include other data, such as recall rate. In some embodiments, the training data may include MRI images that have been labeled (e.g., labeled as containing patterns, features, or characteristics indicative of cancer or other disease; labeled based on image quality; and the like). In some implementations, the training data may include patient data (e.g., accessed from the EHR) paired with manually generated imaging protocols. For example, a hospital database can be accessed to pair labeled patient data (e.g., clinical or diagnosis questions) with the protocols historically chosen by medical personnel for each patient. In some implementations, the training data may include patient data paired with imaging orders (e.g., “brain T₁-weighted image protocol pre and post contrast”).

The method can include assembling training data from hospital record systems using a computer system. This step may include assembling the patient into an appropriate data structure on which the neural network or other machine learning algorithm can be trained. Assembling the training data may include assembling patient data by manually generating imaging protocols, imaging reports, segmentation, diagnosis estimate, and so forth. For instance, assembling the training data may include generating labeled data and including the labeled data in the training data. Labeled data may include MRI imaging protocols associated or paired with various clinical conditions or questions, analyzed images (e.g., segmented images, image reports, and so forth), or other relevant data that have been labeled as belonging to, or otherwise being associated with, one or more different classifications or categories (e.g., images sufficient for clinical question or requiring further scanning, images associated with a disease state). For instance, labeled data may include MRI images that have been segmented to identify a lesion and labeled as having a high level of spiculation. As another example, labeled data may include recall status of an imaging protocol used for a particular clinical question.

### Training the Neural Network(s)

One or more neural networks, such as an LLM, or other suitable machine learning algorithms are trained on the training data, as indicated at step 304. In general, the neural network can be trained by optimizing network parameters (e.g., weights, biases, or both) based on minimizing a loss function. As one non-limiting example, the loss function may be a mean squared error loss function.

Training a neural network may include initializing the neural network, such as by computing, estimating, or otherwise selecting initial network parameters (e.g., weights, biases, or both). During training, an artificial neural network receives the inputs for a training example and generates an output using the bias for each node, and the connections between each node and the corresponding weights. For instance, training data can be input to the initialized neural network, generating output as output imaging pipeline data. The artificial neural network then compares the generated output with the actual output of the training example in order to evaluate the quality of the output imaging pipeline data. For instance, the output imaging pipeline data can be passed to a loss function to compute an error. The current neural network can then be updated based on the calculated error (e.g., using backpropagation methods based on the calculated error). For instance, the current neural network can be updated by updating the network parameters (e.g., weights, biases, or both) in order to minimize the loss according to the loss function. The training continues until a training condition is met. The training condition may correspond to, for example, a predetermined number of training examples being used, a minimum accuracy threshold being reached during training and validation, a predetermined number of validation iterations being completed, and the like. When the training condition has been met (e.g., by determining whether an error threshold or other stopping criterion has been satisfied), the current neural network and its associated network parameters represent the trained neural network. Different types of training processes can be used to adjust the bias values and the weights of the node connections based on the training examples. The training processes may include, for example, gradient descent, Newton's method, conjugate gradient, quasi-Newton, Levenberg-Marquardt, among others.

The artificial neural network can be constructed or otherwise trained based on training data using one or more different learning techniques, such as supervised learning, self-supervised learning, unsupervised learning, reinforcement learning, ensemble learning, active learning, transfer learning, or other suitable learning techniques for neural networks. As an example, supervised learning involves presenting a computer system with example inputs and their actual outputs (e.g., MRI protocol). In these instances, the artificial neural network is configured to learn a general rule or model that maps the inputs to the outputs based on the provided example input-output pairs.

In some implementations, the LLM can be trained on a large group of population data. Such population data can include patient data for a large group of patients. For example, the patient data may include EHR data that outlines a patient's symptoms, imaging orders, and a clinical question posed. In this way, the LLM can be trained to relate the available EHR patient data with a particular clinical question. As another example, the patient data may include a particular clinical question, imaging orders, and protocol details set by a radiologist or imaging technologist. In this way, the LLM can be trained to relate clinical questions to optimized or effective imaging protocols. In some implementations, the training data can further include recall information that characterizes additional scans that were needed or the reason for recall (e.g., poor image quality, insufficient resolution, too much motion). Thus, the LLM can learn to reduce recall rates by correctly assigning an imaging protocol and protocol parameters. As another example, the patient data may include MRI images and radiology reports that can be used to train the LLM to generate a language-based report according to the relevant imaging features, as a radiologist would do. In some implementations, the patient data may further include final diagnoses made by a clinical team that allows the LLM to be trained to estimate a diagnosis based on the acquired MRI images and other EHR data.

In some implementations, training the LLMs can be iterative. For example, a technologist, radiologist, or other clinician can override the automated pipeline results (e.g., by reassigning imaging protocol parameters, by correcting an LLM-generated image report, by ordering additional images). In this way, the LLM can be retrained or adjusted based on the manually-optimized outputs. In some implementations, the LLM can be iteratively trained using LLM-based outputs or other automated outputs. For example, an LLM or physics-based computer module can analyze the images for image quality (resolution, noise level, etc.). Such analysis can be performed while a patient is still undergoing an imaging exam. If the image quality is insufficient, the processor can prompt the LLM to generate a new imaging protocol to improve the image quality. The processor can prompt the scanner to perform such imaging, which can again be analyzed for image quality. Such iterative imaging can be used to further train the LLM for future scanning sessions.

### Storing Neural Network(s) for Later Use

The one or more trained neural networks are then stored for later use, as indicated at step 306. Storing the neural network(s) may include storing network parameters (e.g., weights, biases, or both), which have been computed or otherwise estimated by training the neural network(s) on the training data. Storing the trained neural network(s) may also include storing the particular neural network architecture to be implemented. For instance, data pertaining to the layers in the neural network architecture (e.g., number of layers, type of layers, ordering of layers, connections between layers, hyperparameters for layers) may be stored.

### Use of Foundation Models

In some embodiments, foundation models can be used to additionally or alternatively process the patient data. Foundation models can receive various types of data (e.g., text data, image data, sound data, other 1D, 2D, and/or 3D data types) and generate various types of clinically relevant outputs. For example, the foundation models may generate outputs as predictive scores, imaging protocol parameters, text outputs, image reports, classifications, and so on. As a non-limiting example, text outputs may include answers to questions posed by a clinical user (e.g., medical question answering, tissue parameters or contrast types of interest), interpretive reports of MRI imaging data and/or processed MRI imaging data (e.g., parameter maps, segmented images), or other text-based reports and/or summaries of the input data. Beyond giving diagnostic capabilities, generating reports across temporal and spatial domains with the MRI data and/or processed MRI data is advantageous for identifying if a patient's conditions are improving, worsening, or staying the same and identifying whether a patient requires further imaging to answer the question at hand.

### Large Language Models

As one example, a language model, such as a large language model (LLM), can be used. Use of large language models with patient data involves using machine learning models (e.g., transformer models) to process and analyze patient data, which may include associated clinical text data or medical imaging data. This approach can combine natural language processing (NLP) with computer vision or other multimodal techniques to understand the content of the patient data, medical images, or other physiologic signal data and extract relevant information from them. Advantageously, large language modeling can be applied to patient data or medical images to enable automatic analysis of the input data and to extract clinically relevant information, such as an effective imaging protocol or the presence of neurological conditions or abnormalities. This approach can help healthcare professionals acquire higher quality imaging data with a lower recall rate, make more accurate diagnoses, or provide standardized imaging reports.

One example large language model-based method uses CNNs to extract features from the input patient data, which are then fed into a recurrent neural network (RNN) to generate text descriptions of the patient data. The RNN can also be used to generate imaging protocols, diagnostic questions, or clinical reports based on the input data. Another example approach is to use a transformer-based model, such as a BERT or GPT-4 model, to analyze both the patient data and accompanying text data or protocol data. These models can be pre-trained on large datasets of patient data and associated text and protocol data to improve their accuracy and ability to identify relevant features in the input data.

As a non-limiting example, the patient data can be provided as an input to the LLM. The patient data are first tokenized and converted into a numerical format. The tokenized input data may then be applied to an embedding layer to transform each tokenized input into a high-dimensional vector that captures relationships between the patient data. In some instances, the embedding layer may additionally or alternatively capture semantic relationships between text data and the patient data. The resulting embedded vectors form a one-dimensional sequence that will be input to the LLM. Each element of the embedded vectors corresponds to a token in the tokenized input data.

Any suitable LLM can be used. As one non-limiting example, the LLM may be based on a recurrent layer model, such as a long short term memory (LSTM) model. As another non-limiting example, the LLM may be based on an attention mechanism (e.g., transformer). For instance, the LLM may be based on a generative pre-trained transformer (GPT) model. In still other examples, the LLM may be based on combinations of such model types.

GPT is a type of large language model that is pre-trained on a massive corpus of text data and can generate human-like language. Language models can also be trained on data from other modalities (e.g., patient health records, images, audio recordings, videos, etc.) to enable more diverse capabilities, provide a stronger learning signal, and increase learning speed. Language models can also be formed by fine-tuning a previously trained GPT model. The GPT model is based on the transformer architecture, which allows it to process long sequences of text efficiently. GPT can be used in a wide range of natural language processing (NLP) tasks, including language translation, text summarization, and question answering.

Patient data contain valuable information that can aid in the imaging process, diagnosis, and treatment of various medical conditions. By combining GPT-based language modeling with patient data analysis, the disclosed systems and methods can analyze the input patient data to prescribe imaging protocols and pipelines. The disclosed systems and methods can further analyze generated imaging data to generate textual reports summarizing the findings. For example, a GPT-based model can be trained on a large corpus of generated imaging data and medical reports and then used to generate reports for new imaging data. The generated reports can include information such as the location and size of any abnormalities associated with a respiratory illness, as well as recommendations for further testing, imaging, or treatment.

These LLMs can allow a user to interact with the system at various points within the pipeline. For example, the user can input information, including language prompts that can be interpreted by an LLM, such as one of the LLM agents (e.g., Controller Agent, Protocol Agent, EHR Agent, Analysis Agent, RF Pulse Agent, Gradient Agent, Timing Agent, ADC Agent). In this way, the system can provide a so-called “artificial intelligence (AI) assistant,” “virtual assistant,” or “assistant” that can help to automate the imaging pipeline.

Once a clinical question is set, an LLM or foundational model can be used to generate a tailored imaging protocol. Another LLM or foundation model can be used to analyze the acquired images to provide a report or diagnosis. The models can also be used to determine whether additional imaging is needed in real time. The models can also be used to prescribe a reconstruction pipeline customized to the clinical question of interest and protocol used.

In some instances, the code to these models can be automated or updated using an LLM, GPT, or the like. For example, auto-GPT can be used to write and update its own code and execute scripts. This allows the model to recursively debug, develop, and self-improve. While input data are applied to an auto-GPT-based model, the model can update itself automatically.

As described herein, multimodal language models can be used to receive multiple sources of information to train a language model. In the context of patient data and acquired imaging data, this can mean combining textual information from clinical notes, laboratories, or reports with visual information from imaging data or other sources, including for example previous medical images such as x-rays, CT scans, or MRIs. One approach to multimodal language modeling is to use the GPT architecture, which can be effective in a variety of NLP tasks. The GPT architecture is based on a transformer network, which can learn to model long-range dependencies between words in a sentence. To adapt GPT for multimodal language modeling with patient data or generating imaging data, visual information can be incorporated into the GPT model through pretraining with contrastive learning. This process involves training the model to predict which patient or imaging data and text pairs are related, while also ensuring that unrelated pairs are distinguishable from each other. The resulting model can then be fine-tuned for specific tasks, such as protocol development, imaging data captioning, medical report generation, or disease diagnosis. For example, a model trained on clinical notes and imaging protocol data can be fine-tuned to generate effective imaging protocols. As another example, a model trained on imaging data and radiological reports can be fine-tuned to generate reports to predict the presence of certain neurological conditions (e.g., brain injuries, neurological diseases, etc.) based on the imaging data and patient history. In some implementations, the LLMs can also be fine-tuned using Low Rank Adaptation (LoRA). In some implementations, the LLMs can be trained or fine-tuned using retrieval-augmented generation (RAG).

### Visual Transformers

As another example, a visual transformer-based model can be used. In these instances, higher-dimensional inputs can be provided to the underlying foundation model. For example, the generated imaging data and/or processed imaging data may be tokenized and embedded, as described above, into a 2D or other N-dimensional (N≥2) vector, matrix, or tensor. These higher dimensional input data can then be applied to a suitable foundation model, such as a visual transformer-based model.

Vision-language processing can be improved by using paired samples sharing semantics. For instance, given an image and text pair, the text should describe the image with minimal extraneous detail. Without knowledge of this initial image, temporal information in the text modality (e.g., “condition is improving,” “condition is worsening,” “condition is stable”) could pertain to any image including “condition”, creating vagueness during contrastive training. Vision-language processing implementations can, in some instances, assume alignment between single images and reports, removing temporal content from reports in training data to prevent hallucinations in downstream report generation.

In other implementations of vision-language processing, temporal information can provide complementary self-supervision by using an existing structure without requiring additional data. Rather than treating all image-report pairs in the dataset as independent, temporal correlations can be used by making previous images available for comparison to a given report. A temporal vision-language processing pretraining framework can be learned from this structure. In some implementations, a multi-image encoder that can handle the absence of previous images and potential spatial misalignment between images across time can be used in this vision-language processing. Prior images can be accounted for where available, thereby removing cross-modal ambiguity. Linking multiple images or datasets has the advantage of improving image and text models and performance on temporal image classification and report generation. Prefixing the prior report can significantly improve performance. When available during training and fine-tuning, earlier images and labels can also be accounted for.

As one non-limiting example, a convolutional neural network (CNN) or LLM visual transformer hybrid multi-image encoder can be trained jointly with a text model. The hybrid model can provide improved processing for tasks in both single-image and multi-image structures, achieving performance on disease progression classification, phrase grounding, and document generation, while offering consistent modifications on disease category and sentence-similarity tasks. The similarity between the generated imaging data, patient data, or other signals and text embeddings can be computed to obtain probabilities, which can be used to classify the various categories of the imaging data and then reported textually. The outputs can also include output imaging pipeline data indicating risk categories of no risk, low risk, medium risk, and high risk.

### EXAMPLES

### Example 1

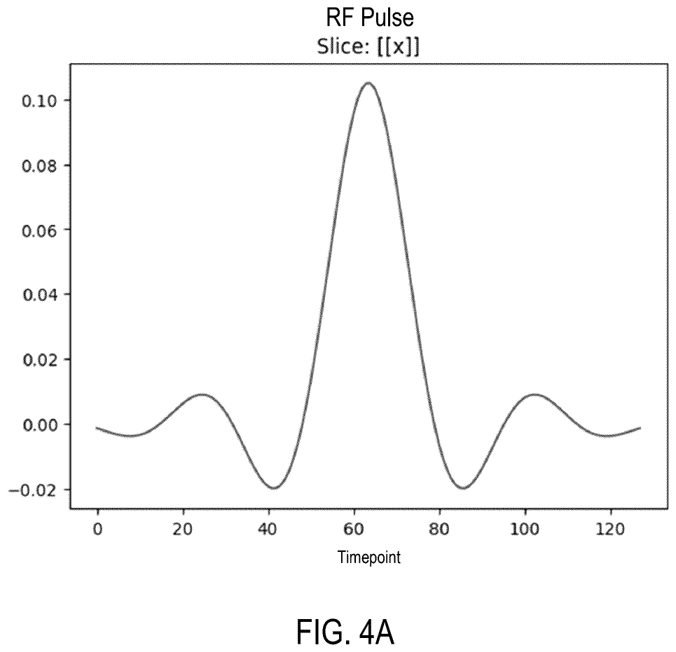

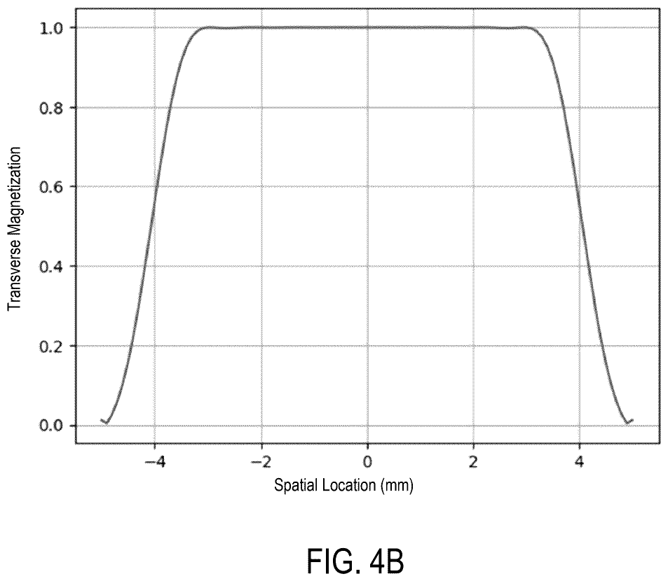

As a non-limiting example, an LLM-based RF pulse agent was trained to generate RF pulse event blocks as prompted by a user. A full transcript between the user and LLM agent, referred to as “assistant,” is provided below, and the corresponding RF pulse and slice profile are shown in FIGS. 4A and 4B. The user asked the LLM agent to produce an excitation pulse with 128 timepoints and a time bandwidth product of 8. The LLM agent interpreted the request and generated an RF pulse waveform, as shown in the transcript below and in FIG. 4A. The user further asked the LLM agent to simulate the output RF pulse waveform for a 10 mm slice thickness. The LLM agent produces a transverse magnetization profile across the 10 mm slice, as shown in the transcript below and FIG. 4B.

### Example Transcript:

### Begin Transcript

“user: Design an excitation pulse with 128 timepoints and time bandwidth 8
assistant: The designed excitation RF pulse with 128 time points and a time-bandwidth product of 8 has been successfully created. The complex-valued array containing the RF pulse values is as follows:
“‘json
[−0.0013110928452310716, −0.0017585985284948368, −0.0021858167305739656, −0.0026150861445159183, −0.003005108772348101, −0.0033443364835216054, −0.0036018296576841324, −0.0037602264819196264, −0.0037946470498985167, −0.0036902708584237004, −0.0034308473784139043, −0.003009782094191002, −0.0024229237471746976, −0.001676062304313623, −0.0007797656396662193, 0.00024553029010515034, 0.0013737628021452304, 0.0025699902327973004, 0.0037943168999973235, 0.005000128630934244, 0.006137993564102444, 0.0071550854889573376, 0.007999129561032318, 0.008618680486726208, 0.008967011065631744, 0.00900285221264548, 0.008693981062966072, 0.008017996665432183, 0.006965294498050934, 0.005539467164953449, 0.003759371828766686, 0.0016588292481404486, −0.0007124133053292578, −0.003289886139982, −0.005994625894206295, −0.008735362179151813, −0.011409928289372923, −0.013908413503561179, 0.01611564503224698, −0.017915149116995814, −0.019192426145045252, −0.01983933098886342, −0.01975761443785406, −0.018863095025795494, −0.017088728395856276, −0.014387865223434205, −0.010736232508443903, −0.0061338420828545145, −0.0006057079648802605, 0.005797460926788699, 0.0129994452159526, 0.020898526785231783, 0.029368081525503565, 0.03825700765708041, 0.04739085161383724, 0.05657329543847418, 0.06558903407375848, 0.07420800411768291, 0.08219189274997389, 0.08930276013988905, 0.09531390735771023, 0.10002219402056126, 0.10326084987551983, 0.10491109115648972, 0.10491109120976191, 0.10326085003301556, 0.10002219427673609, 0.09531390770444506, 0.08930276056722444, 0.08219189325007902, 0.07420800468243463, 0.06558903469065737, 0.05657329609572107, 0.047390852302689704, 0.03825700836904336, 0.029368082251983108, 0.020898527517440094, 0.012999445949064877, 0.005797461653524088, −0.0006057072514255078, −0.006133841386834762, −0.010736231834668908, −0.01438786457546706, −0.017088727776718292, −0.018863094438889942, −0.019757613887507918, −0.019839330476022706, −0.01919242566956902, −0.017915148679751757, −0.016115644635691123, −0.0139084131488902, −0.011409927976895837, −0.008735361907352416, −0.005994625662368359, −0.0032898859459687202, −0.0007124131471137089, 0.0016588293699714585, 0.003759371914562303, 0.0055394672132003326, 0.006965294507130344, 0.008017996633406792, 0.008693980989398881, 0.009002852096782766, 0.008967010909200913, 0.008618680291134637, 0.007999129327630762, 0.007155085219094603, 0.0061379932580292096, 0.005000128291566774, 0.003794316530930644, 0.0025699898350968102, 0.0013737623785852935, 0.00024552984284733226, −0.0007797661054634447, −0.00167606278277889, −0.0024229242293265624, −0.003009782572260768, −0.003430847847607431, −0.003690271311996845, −0.003794647479281922, −0.0037602268773632755, −0.003601830012881879, −0.0033443367907296812, −0.003005109022486721, −0.0026150863295014196, −0.00218581684511247, −0.0017585985709532663, −0.0013110928106168523]
’”

This pulse should provide the required excitation profile for your MRI application.
user: Simulate the designed RF pulse over 10 mm slice thickness
assistant: The simulation of the designed excitation RF pulse over a 10 mm slice thickness has been completed. The resulting transverse magnetization profile is as follows:

The full range of spatial locations from −5 mm to +5 mm and their corresponding simulated transverse magnetization values are indicated, providing a profile for the 10 mm slice thickness. At the center, the magnetization is highest, near unity, indicating effective excitation at that location, while it tapers off at the edges of the slice.”

### End Transcript

### Example 2

One example implementation consists of a Controller Agent managing three specialized worker agents: an EHR Agent for database queries, a Protocol Agent for MR protocol generation, and a Sequence Agent for creating pulse sequences, to enable automatic customization of MR protocols based on patient EHR data.

### Methods

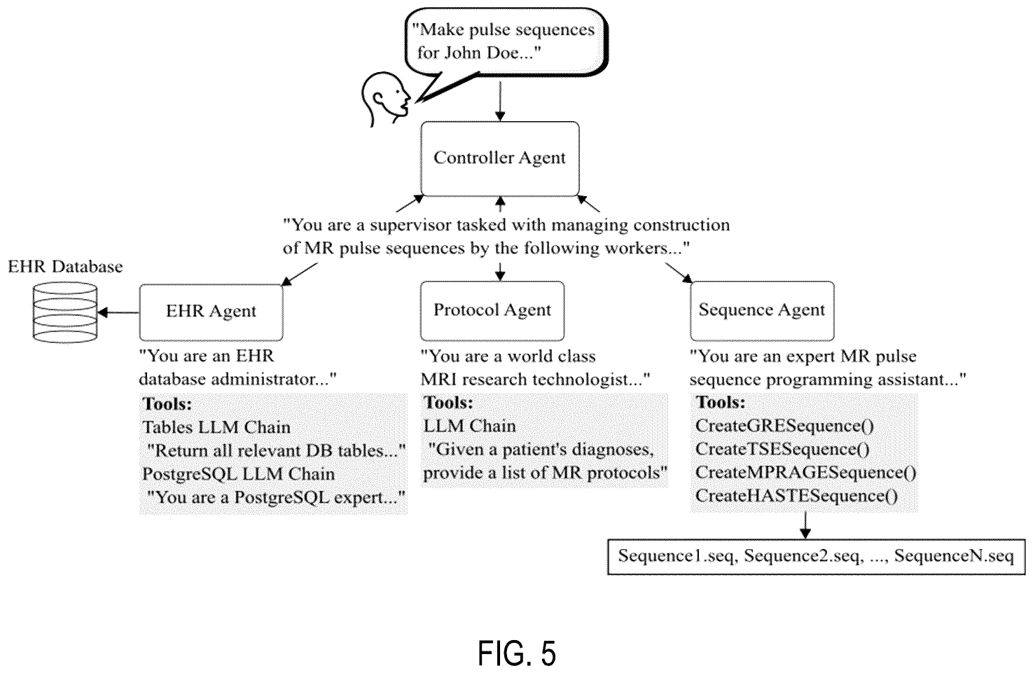

The multi-agent system was implemented in LangGraph following a supervisor model, with each agent having its own prompt, LLM and custom software tools, as shown in FIG. 5.

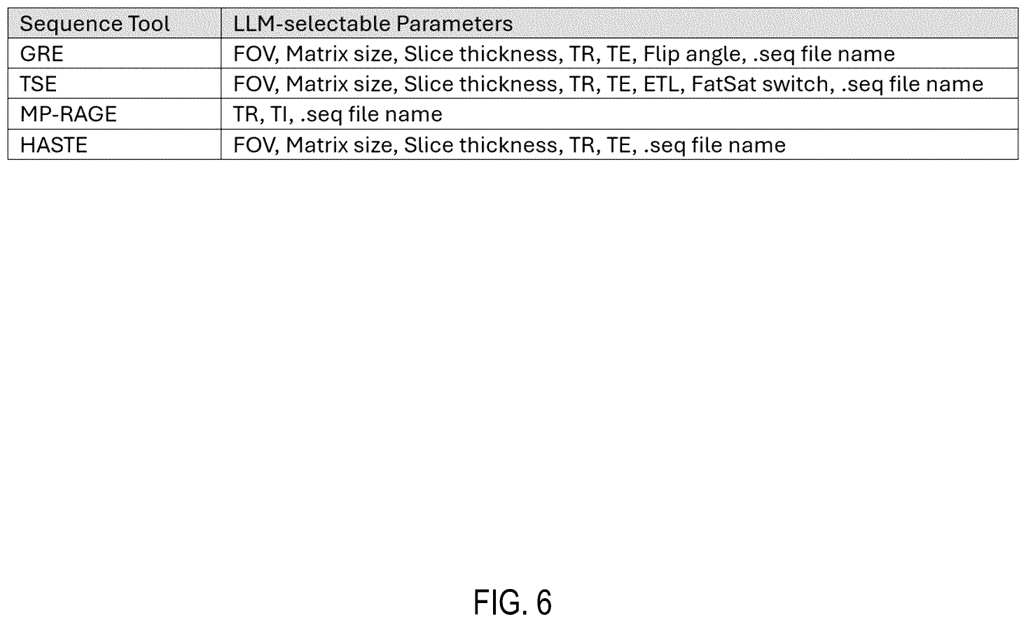

The Controller Agent serves as the sole human-facing interface and supervises the EHR Agent, Protocol Agent, and Sequence Agent workers. When presented with a human prompt, the Controller Agent autonomously and iteratively selects the next worker agent to call until the Controller Agent determines that the desired pulse sequences have been created, and it stops the execution chain. We simulated a hospital database using MIMIC-IV, containing EHR from 180,733 patients in PostgreSQL. The system comprises three LangChain ReActagents: the EHR Agent executes JSON formatted SQL queries based on natural language input; the Protocol Agent uses a prompt-LLM-parser chain to prescribe imaging protocols; and the Sequence Agent autonomously generates scanner-ready sequences using Pulseq tools for GRE, TSE, HASTE, and MP-RAGE with optimized parameters, as shown in FIG. 6.

These tools are Pulseq sequence implementations that take sequence parameters as input and write the resulting .seq files to disk. All agents utilized GPT-40 with autonomous reasoning through Thought/Action/Observation cycles, without human-coded decision trees. The multi-agent system was tested on three patients with abdomen (ID 10085005), brain (ID 17940697) and knee-related (ID 10713795) diagnoses in the MIMIC-IV database. The Pulseq sequences generated for the brain patient were executed in a volunteer on a 3 T whole-body scanner (Vida, Siemens Healthineers) with IRB approval.

### Results

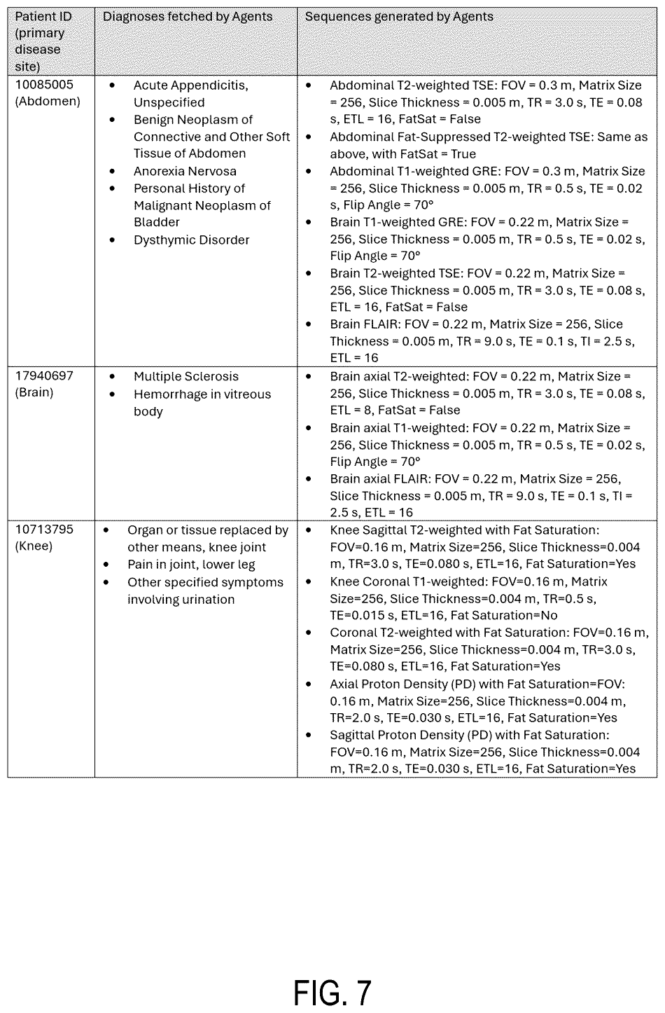

FIG. 7 shows multi-agent system's outputs for three clinical cases (abdomen, brain, and knee) in the MIMIC-IV database. For each case, the system successfully retrieved relevant diagnoses (middle column) from the EHR database and generated corresponding customized pulse sequences (right column).

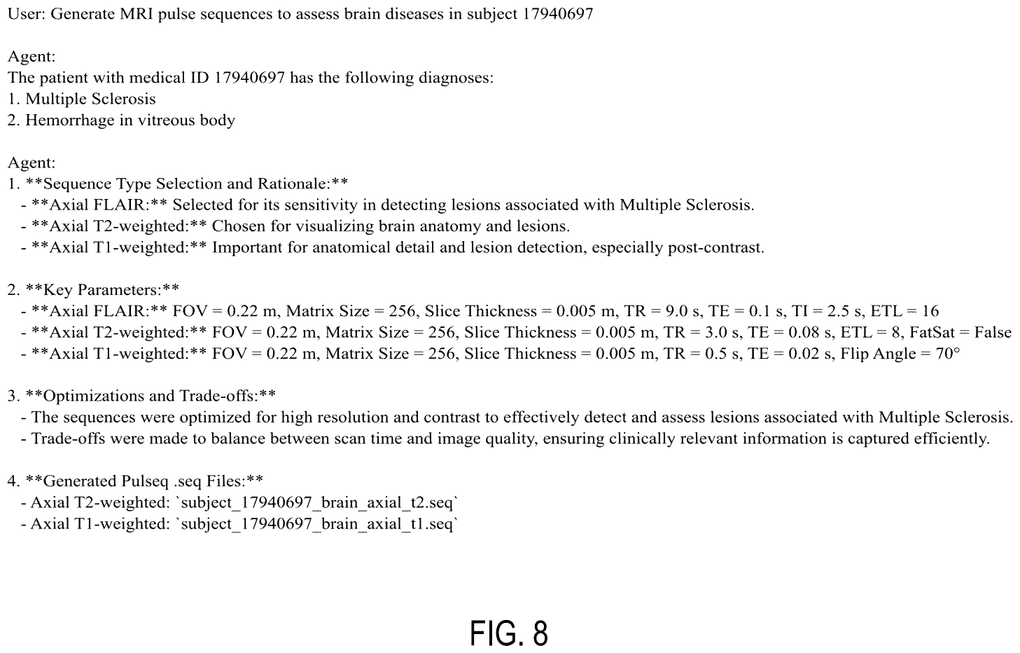

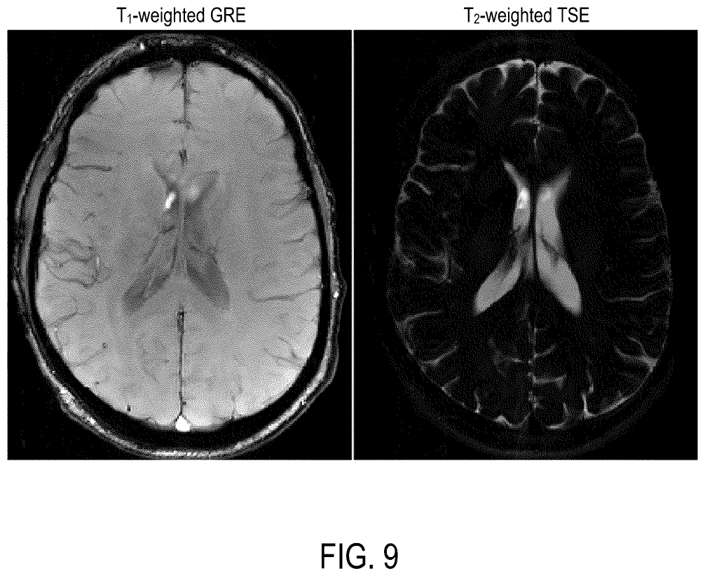

FIG. 8 shows the agents' key responses when prompted with a prompt as: “Generate MRI pulse sequences to assess brain diseases in subject 17940697”. The EHR Agent was successfully able to retrieve the ICD codes from the MIMIC-IV database and convert them into diagnoses text for patient with ID 17940697. The diagnoses were manually verified against the values in the MIMIC-IV database. The Protocol Agent prescribed a head exam comprising T₁-weighted, T₂-weighted, and T₂-FLAIR protocols with rationale for the selection. Further, the Sequence Agent successfully created T₁-weighted GRE, and T₂-weighted TSE Pulseq sequences with the parameters needed to achieve the desired contrast. FIG. 9 shows images acquired with pulse sequences generated by the multi-agent system for the brain patient (ID 17940697). The sequences successfully produced images with the desired T₁- and T₂-weighted contrasts for brain tissue assessment.

### Example Systems

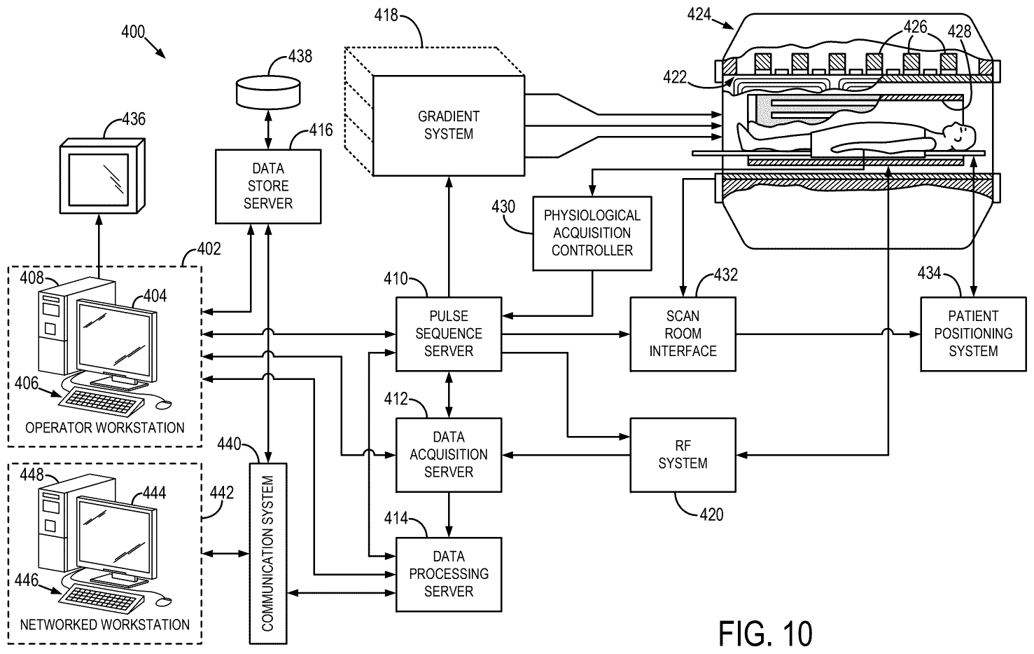

Referring particularly now to FIG. 10, an example of an MRI system 400 that can implement the methods described herein is illustrated. The MRI system 400 includes an operator workstation 402 that may include a display 404, one or more input devices 406 (e.g., a keyboard, a mouse), and a processor 408. The processor 408 may include a commercially available programmable machine running a commercially available operating system. The operator workstation 402 provides an operator interface that facilitates entering scan parameters into the MRI system 400. The operator workstation 402 may be coupled to different servers, including, for example, a pulse sequence server 410, a data acquisition server 412, a data processing server 414, and a data store server 416. The operator workstation 402 and the servers 410, 412, 414, and 416 may be connected via a communication system 440, which may include wired or wireless network connections.

The MRI system 400 also includes a magnet assembly 424 that includes a polarizing magnet 426, which may be a low-field magnet. The MRI system 400 may optionally include a whole-body RF coil 428 and a gradient system 418 that controls a gradient coil assembly 422.

The pulse sequence server 410 functions in response to instructions provided by the operator workstation 402 to operate a gradient system 418 and a radiofrequency (“RF”) system 420. Gradient waveforms for performing a prescribed scan are produced and applied to the gradient system 418, which then excited gradient coils in an assembly 422 to produce the magnetic field gradients (e.g., Gx, Gy, and Gz) that can be used for spatially encoding magnetic resonance signals. The gradient coil assembly 422 forms part of a magnet assembly 424 that includes a polarizing magnet 426 and a whole-body RF coil 428.

RF waveforms are applied by the RF system 420 to the RF coil 428, or a separate local coil to perform the prescribed magnetic resonance pulse sequence. Responsive magnetic resonance signals detected by the RF coil 428, or a separate local coil, are received by the RF system 420. The responsive magnetic resonance signals may be amplified, demodulated, filtered, and digitized under direction of commands produced by the pulse sequence server 410. The RF system 420 includes an RF transmitter for producing a wide variety of RF pulses used in MRI pulse sequences. The RF transmitter is responsive to the prescribed scan and direction from the pulse sequence server 410 to produce RF pulses of the desired frequency, phase, and pulse amplitude waveform. The generated RF pulses may be applied to the whole-body RF coil 428 or to one or more local coils or coil arrays.

The RF system 420 also includes one or more RF receiver channels. An RF receiver channel includes an RF preamplifier that amplifies the magnetic resonance signal received by the coil 428 to which it is connected, and a detector that detects and digitizes the/and Q quadrature components of the received magnetic resonance signal. The magnitude of the received magnetic resonance signal may, therefore, be determined at a sampled point by the square root of the sum of the squares of the I and Q components:

$$M = \sqrt{( {I}^{2} + {Q}^{2} )}$$

and the phase of the received magnetic resonance signal may also be determined according to the following relationship:

$$ϕ = {tan}^{- 1} ( \frac{Q}{I} )$$

The pulse sequence server 410 may receive patient data from a physiological acquisition controller 430. By way of example, the physiological acquisition controller 430 may receive signals from a number of different sensors connected to the patient, including electrocardiograph (“ECG”) signals from electrodes, or respiratory signals from a respiratory bellows or other respiratory monitoring devices. These signals may be used by the pulse sequence server 410 to synchronize, or “gate,” the performance of the scan with the subject's heartbeat or respiration.

The pulse sequence server 410 may also connect to a scan room interface circuit 432 that receives signals from various sensors associated with the condition of the patient and the magnet system. Through the scan room interface circuit 432, a patient positioning system 434 can receive commands to move the patient to desired positions during the scan.

The digitized magnetic resonance signal samples produced by the RF system 420 are received by the data acquisition server 412. The data acquisition server 412 operates in response to instructions downloaded from the operator workstation 402 to receive the real-time magnetic resonance data and provide buffer storage, so that data are not lost by data overrun. In some scans, the data acquisition server 412 passes the acquired magnetic resonance data to the data processor server 414. In scans that require information derived from acquired magnetic resonance data to control the further performance of the scan, the data acquisition server 412 may be programmed to produce such information and convey it to the pulse sequence server 410. For example, during pre-scans, magnetic resonance data may be acquired and used to calibrate the pulse sequence performed by the pulse sequence server 410. As another example, navigator signals may be acquired and used to adjust the operating parameters of the RF system 420 or the gradient system 418, or to control the view order in which k-space is sampled. In still another example, the data acquisition server 412 may also process magnetic resonance signals used to detect the arrival of a contrast agent in a magnetic resonance angiography (“MRA”) scan. For example, the data acquisition server 412 may acquire magnetic resonance data and processes it in real-time to produce information that is used to control the scan.

The data processing server 414 receives magnetic resonance data from the data acquisition server 412 and processes the magnetic resonance data in accordance with instructions provided by the operator workstation 402. Such processing may include, for example, reconstructing two-dimensional or three-dimensional images by performing a Fourier transformation of raw k-space data, performing other image reconstruction algorithms (e.g., iterative or backprojection reconstruction algorithms), applying filters to raw k-space data or to reconstructed images, generating functional magnetic resonance images, or calculating motion or flow images.

Images reconstructed by the data processing server 414 are conveyed back to the operator workstation 402 for storage. Real-time images may be stored in a data base memory cache, from which they may be output to operator display 402 or a display 436. Batch mode images or selected real time images may be stored in a host database on disc storage 438. When such images have been reconstructed and transferred to storage, the data processing server 414 may notify the data store server 416 on the operator workstation 402. The operator workstation 402 may be used by an operator to archive the images, produce films, or send the images via a network to other facilities.

The MRI system 400 may also include one or more networked workstations 442. For example, a networked workstation 442 may include a display 444, one or more input devices 446 (e.g., a keyboard, a mouse), and a processor 448. The networked workstation 442 may be located within the same facility as the operator workstation 402, or in a different facility, such as a different healthcare institution or clinic.

The networked workstation 442 may gain remote access to the data processing server 414 or data store server 416 via the communication system 440. Accordingly, multiple networked workstations 442 may have access to the data processing server 414 and the data store server 416. In this manner, patient data, magnetic resonance data, reconstructed images, or other data may be exchanged between the data processing server 414 or the data store server 416 and the networked workstations 442, such that the data or images may be remotely processed by a networked workstation 442.

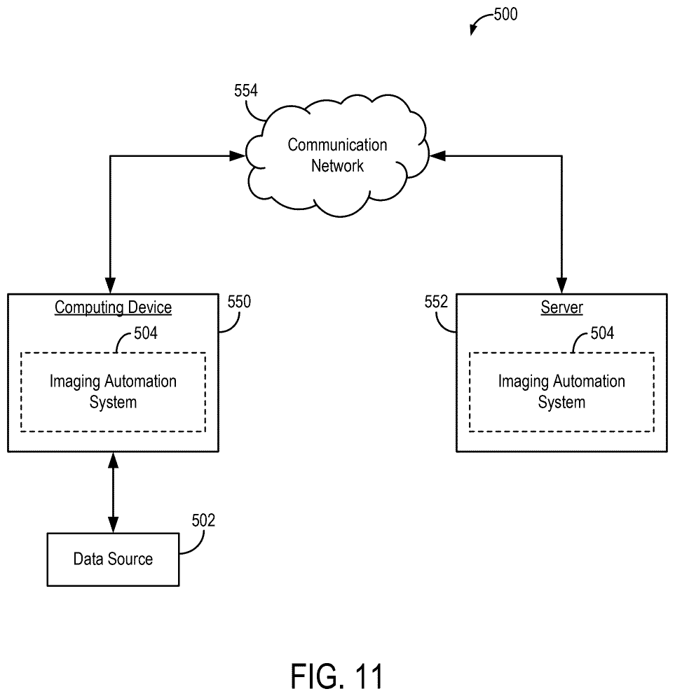

Referring now to FIG. 11, an example of an MRI system 500 is shown, which may be used in accordance with some aspects of the systems and methods described in the present disclosure. As shown in FIG. 11, a computing device 550 can receive one or more types of data (e.g., patient data, signal evolution data, k-space data, receiver coil sensitivity data) from data source 502. In some configurations, computing device 550 can execute at least a portion of an imaging automation system 504 to reconstruct images from magnetic resonance data (e.g., k-space data) acquired using an LLM-generated imaging protocol. In some configurations, the imaging automation system 504 can implement an automated pipeline to provide MRI images, quantitative parameter maps, MRF maps, MRF synthetic images, image analysis reports, imaging protocols, reconstruction pipelines, etc.

Additionally or alternatively, in some configurations, the computing device 550 can communicate information about data received from the data source 502 to a server 552 over a communication network 554, which can execute at least a portion of the imaging automation system 504. In such configurations, the server 552 can return information to the computing device 550 (and/or any other suitable computing device) indicative of an output of the imaging automation system 504.

In some configurations, computing device 550 and/or server 552 can be any suitable computing device or combination of devices, such as a desktop computer, a laptop computer, a smartphone, a tablet computer, a wearable computer, a server computer, a virtual machine being executed by a physical computing device, and so on. The computing device 550 and/or server 552 can also reconstruct images from the data.

In some configurations, data source 502 can be any suitable source of data (e.g., EHR database, measurement data, images reconstructed from measurement data, processed image data), such as an MRI system, another computing device (e.g., a server storing measurement data, images reconstructed from measurement data, processed image data), and so on. In some configurations, data source 502 can be local to computing device 550. For example, data source 502 can be incorporated with computing device 550 (e.g., computing device 550 can be configured as part of a device for measuring, recording, estimating, acquiring, or otherwise collecting or storing data). As another example, data source 502 can be connected to computing device 550 by a cable, a direct wireless link, and so on. Additionally or alternatively, in some configurations, data source 502 can be located locally and/or remotely from computing device 550, and can communicate data to computing device 550 (and/or server 552) via a communication network (e.g., communication network 554).

In some configurations, communication network 554 can be any suitable communication network or combination of communication networks. For example, communication network 554 can include a Wi-Fi network (which can include one or more wireless routers, one or more switches, etc.), a peer-to-peer network (e.g., a Bluetooth network), a cellular network (e.g., a 3G network, a 4G network, etc., complying with any suitable standard, such as CDMA, GSM, LTE, LTE Advanced, WiMAX, etc.), other types of wireless network, a wired network, and so on. In some configurations, communication network 554 can be a local area network, a wide area network, a public network (e.g., the Internet), a private or semi-private network (e.g., a corporate or university intranet), any other suitable type of network, or any suitable combination of networks. Communications links shown in FIG. 11 can each be any suitable communications link or combination of communications links, such as wired links, fiber optic links, Wi-Fi links, Bluetooth links, cellular links, and so on.

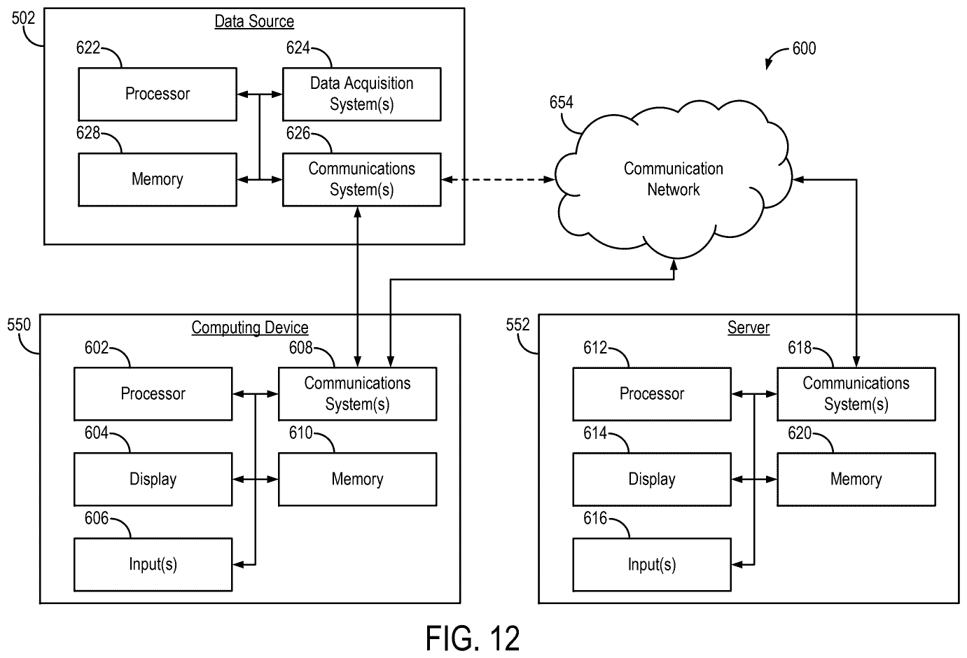

Referring now to FIG. 12, an example of hardware 600 that can be used to implement data source 502, computing device 550, and server 552 in accordance with some configurations of the systems and methods described in the present disclosure is shown.

As shown in FIG. 12, in some configurations, computing device 550 can include a processor 602, a display 604, one or more inputs 606, one or more communication systems 608, and/or memory 610. In some configurations, processor 602 can be any suitable hardware processor or combination of processors, such as a central processing unit (“CPU”), a graphics processing unit (“GPU”), and so on. In some configurations, display 604 can include any suitable display devices, such as a liquid crystal display (“LCD”) screen, a light-emitting diode (“LED”) display, an organic LED (“OLED”) display, an electrophoretic display (e.g., an “e-ink” display), a computer monitor, a touchscreen, a television, and so on. In some configurations, inputs 606 can include any suitable input devices and/or sensors that can be used to receive user input, such as a keyboard, a mouse, a touchscreen, a microphone, and so on.

In some configurations, communications systems 608 can include any suitable hardware, firmware, and/or software for communicating information over communication network 554 and/or any other suitable communication networks. For example, communications systems 608 can include one or more transceivers, one or more communication chips and/or chip sets, and so on. In a more particular example, communications systems 608 can include hardware, firmware, and/or software that can be used to establish a Wi-Fi connection, a Bluetooth connection, a cellular connection, an Ethernet connection, and so on.

In some configurations, memory 610 can include any suitable storage device or devices that can be used to store instructions, values, data, or the like, that can be used, for example, by processor 602 to present content using display 604, to communicate with server 552 via communications system(s) 608, and so on. Memory 610 can include any suitable volatile memory, non-volatile memory, storage, or any suitable combination thereof. For example, memory 610 can include random-access memory (“RAM”), read-only memory (“ROM”), electrically programmable ROM (“EPROM”), electrically erasable ROM (“EEPROM”), other forms of volatile memory, other forms of non-volatile memory, one or more forms of semi-volatile memory, one or more flash drives, one or more hard disks, one or more solid state drives, one or more optical drives, and so on. In some configurations, memory 610 can have encoded thereon, or otherwise stored therein, a computer program for controlling operation of computing device 550. In such configurations, processor 602 can execute at least a portion of the computer program to present content (e.g., images, user interfaces, graphics, tables), receive content from server 552, transmit information to server 552, and so on. For example, the processor 602 and the memory 610 can be configured to perform the methods described herein.

In some configurations, server 552 can include a processor 612, a display 614, one or more inputs 616, one or more communications systems 618, and/or memory 620. In some configurations, processor 612 can be any suitable hardware processor or combination of processors, such as a CPU, a GPU, and so on. In some configurations, display 614 can include any suitable display devices, such as an LCD screen, LED display, OLED display, electrophoretic display, a computer monitor, a touchscreen, a television, and so on. In some configurations, inputs 616 can include any suitable input devices and/or sensors that can be used to receive user input, such as a keyboard, a mouse, a touchscreen, a microphone, and so on.

In some configurations, communications systems 618 can include any suitable hardware, firmware, and/or software for communicating information over communication network 554 and/or any other suitable communication networks. For example, communications systems 618 can include one or more transceivers, one or more communication chips and/or chip sets, and so on. In a more particular example, communications systems 618 can include hardware, firmware, and/or software that can be used to establish a Wi-Fi connection, a Bluetooth connection, a cellular connection, an Ethernet connection, and so on.

In some configurations, memory 620 can include any suitable storage device or devices that can be used to store instructions, values, data, or the like, that can be used, for example, by processor 612 to present content using display 614, to communicate with one or more computing devices 550, and so on. Memory 620 can include any suitable volatile memory, non-volatile memory, storage, or any suitable combination thereof. For example, memory 620 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some configurations, memory 620 can have encoded thereon a server program for controlling operation of server 552. In such configurations, processor 612 can execute at least a portion of the server program to transmit information and/or content (e.g., data, images, a user interface) to one or more computing devices 550, receive information and/or content from one or more computing devices 550, receive instructions from one or more devices (e.g., a personal computer, a laptop computer, a tablet computer, a smartphone), and so on.

In some configurations, the server 552 is configured to perform the methods described in the present disclosure. For example, the processor 612 and memory 620 can be configured to perform the methods described herein.

In some configurations, data source 502 can include a processor 622, one or more data acquisition systems 624, one or more communications systems 626, and/or memory 628. In some configurations, processor 622 can be any suitable hardware processor or combination of processors, such as a CPU, a GPU, and so on. In some configurations, the one or more data acquisition systems 624 are generally configured to acquire data, images, or both, and can include an MRI system. Additionally or alternatively, in some configurations, the one or more data acquisition systems 624 can include any suitable hardware, firmware, and/or software for coupling to and/or controlling operations of an MRI system. In some configurations, one or more portions of the data acquisition system(s) 624 can be removable and/or replaceable.

Note that, although not shown, data source 502 can include any suitable inputs and/or outputs. For example, data source 502 can include input devices and/or sensors that can be used to receive user input, such as a keyboard, a mouse, a touchscreen, a microphone, a trackpad, a trackball, and so on. As another example, data source 502 can include any suitable display devices, such as an LCD screen, an LED display, an OLED display, an electrophoretic display, a computer monitor, a touchscreen, a television, etc., one or more speakers, and so on.

In some configurations, communications systems 626 can include any suitable hardware, firmware, and/or software for communicating information to computing device 550 (and, in some configurations, over communication network 554 and/or any other suitable communication networks). For example, communications systems 626 can include one or more transceivers, one or more communication chips and/or chip sets, and so on. In a more particular example, communications systems 626 can include hardware, firmware, and/or software that can be used to establish a wired connection using any suitable port and/or communication standard (e.g., VGA, DVI video, USB, RS-232, etc.), Wi-Fi connection, a Bluetooth connection, a cellular connection, an Ethernet connection, and so on.

In some configurations, memory 628 can include any suitable storage device or devices that can be used to store instructions, values, data, or the like, that can be used, for example, by processor 622 to control the one or more data acquisition systems 624, and/or receive data from the one or more data acquisition systems 624; to generate images from data; present content (e.g., data, images, a user interface) using a display; communicate with one or more computing devices 550; and so on. Memory 628 can include any suitable volatile memory, non-volatile memory, storage, or any suitable combination thereof. For example, memory 628 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some configurations, memory 628 can have encoded thereon, or otherwise stored therein, a program for controlling operation of medical image data source 502. In such configurations, processor 622 can execute at least a portion of the program to generate images, transmit information and/or content (e.g., data, images, a user interface) to one or more computing devices 550, receive information and/or content from one or more computing devices 550, receive instructions from one or more devices (e.g., a personal computer, a laptop computer, a tablet computer, a smartphone, etc.), and so on.

In some configurations, any suitable computer-readable media can be used for storing instructions for performing the functions and/or processes described herein. For example, in some configurations, computer-readable media can be transitory or non-transitory. For example, non-transitory computer-readable media can include media such as magnetic media (e.g., hard disks, floppy disks), optical media (e.g., compact discs, digital video discs, Blu-ray discs), semiconductor media (e.g., RAM, flash memory, EPROM, EEPROM), any suitable media that is not fleeting or devoid of any semblance of permanence during transmission, and/or any suitable tangible media. As another example, transitory computer-readable media can include signals on networks, in wires, conductors, optical fibers, circuits, or any suitable media that is fleeting and devoid of any semblance of permanence during transmission, and/or any suitable intangible media.

As used herein in the context of computer implementation, unless otherwise specified or limited, the terms “component,” “system,” “module,” “controller,” “framework,” and the like are intended to encompass part or all of computer-related systems that include hardware, software, a combination of hardware and software, or software in execution. For example, a component may be, but is not limited to being, a processor device, a process being executed (or executable) by a processor device, an object, an executable, a thread of execution, a computer program, or a computer. By way of illustration, both an application running on a computer and the computer can be a component. One or more components (or system, module, and so on) may reside within a process or thread of execution, may be localized on one computer, may be distributed between two or more computers or other processor devices, or may be included within another component (or system, module, and so on).

In some implementations, devices or systems disclosed herein can be utilized or installed using methods embodying aspects of the disclosure. Correspondingly, description herein of particular features, capabilities, or intended purposes of a device or system is generally intended to inherently include disclosure of a method of using such features for the intended purposes, a method of implementing such capabilities, and a method of installing disclosed (or otherwise known) components to support these purposes or capabilities. Similarly, unless otherwise indicated or limited, discussion herein of any method of manufacturing or using a particular device or system, including installing the device or system, is intended to inherently include disclosure, as embodiments of the disclosure, of the utilized features and implemented capabilities of such device or system.

As used herein, the phrase “at least one of A, B, and C” means at least one of A, at least one of B, and/or at least one of C, or any one of A, B, or C or combination of A, B, or C. A, B, and C are elements of a list, and A, B, and C may be anything contained in the Specification.

The present disclosure has described one or more preferred embodiments, and it should be appreciated that many equivalents, alternatives, variations, and modifications, aside from those expressly stated, are possible and within the scope of the invention.

1. A system for automating a magnetic resonance imaging (MRI) pipeline for a patient, the system comprising:
one or more processors configured to:
receive information comprising patient data;
access one or more trained large language models (LLMs); and
apply the patient data to the one or more trained LLMs to generate an MRI protocol comprising one or more pulse sequences with pulse sequence parameters; and
an MRI system configured to acquire imaging data from the patient according to the MRI protocol.

2. The system of claim 1, wherein the MRI protocol is a magnetic resonance fingerprinting (MRF) protocol.

3. The system of claim 2, wherein the pulse sequence parameters comprise a varying parameter that changes throughout the pulse sequence, the varying parameter comprising at least one of a flip angle, an echo time, or a repetition time; and
wherein changes of the varying parameter are determined by the one or more trained LLMs based on the patient data.

4. The system of claim 1, wherein the one or more processors are further configured to generate an event table of the one or more pulse sequences using physics-based methods, the event table comprising at least one of radiofrequency (RF) pulse blocks, gradient waveform blocks, analog to digital converter blocks, or timing parameters.

5. The system of claim 1, wherein the one or more processors are further configured to:
receive the imaging data from the MRI system;
apply the imaging data to one of the one or more LLMs to generate a reconstruction pipeline; and
apply the reconstruction pipeline to the imaging data to generate images.

6. The system of claim 5, wherein the reconstruction pipeline is configured to generate quantitative tissue parameter maps.

7. The system of claim 5, wherein the one or more processors are further configured to:
apply the images to one of the one or more LLMs to analyze the images and generate a report based on the images.

8. The system of claim 7, wherein the one or more processors are further configured to:
apply the report to the one or more LLMs to update at least one of the MRI protocol or the reconstruction pipeline.

9. The system of claim 1, wherein the patient data comprises a patient identification; and wherein, when applying the patient data to the one or more LLMs to generate the MRI protocol, the one or more processors are configured to:
apply the patient identification to a first of the one or more LLMs to determine clinically relevant information about the patient; and
apply the clinically relevant information about the patient to a second of the one or more LLMs to generate the MRI protocol.

10. The system of claim 9, wherein the one or more processors are configured to apply the patient identification to the first of the one or more LLMs using an electronic health record agent that has access to an electronic health record database.

11. The system of claim 1, wherein the one or more processors are configured to:
cause an electronic health record (EHR) agent to apply the patient data to one of the one or more LLMs to determine clinically relevant patient information;
cause a Protocol Agent to apply the clinically relevant patient information to one of the one or more LLMs to determine the pulse sequence type;
cause a Sequence Agent to apply the pulse sequence type to one of the one or more LLMs to determine pulse sequence parameters; and
oversee actions of the EHR Agent, Protocol Agent, and Sequence Agent, using a Controller Agent.

12. The system of claim 11, wherein the Controller Agent is in communication with a user to receive the patient data.

13. The system of claim 11, wherein the one or more processors are further configured to:
generate an event table based on the pulse sequence type and pulse sequence parameters;
communicate the event table to an MRI system;
receive imaging data acquired by the MRI system based on the event table;
cause a Reconstruction Agent to apply the imaging data to one of the one or more LLMs to generate a reconstruction pipeline;
apply the reconstruction pipeline to the imaging data to generate images;
cause an Analysis Agent to apply the images to one of the one or more LLMs to generate a report;
cause the Controller Agent to communicate the report to the user; and
oversee actions of the Reconstruction Agent and the Analysis Agent using the Controller Agent.

14. The system of claim 1, wherein the pulse sequence parameters comprise at least one of field of view size, matrix size, slice thickness, slice geometry, repetition time, echo time, flip angle, fat saturation switch, inversion time, or diffusion weighting.

15. The system of claim 1, wherein the one or more processors are further configured to determine whether the MRI protocol fits within hardware and safety limits of an MRI system and regenerate the MRI protocol if the MRI protocol does not fit within the hardware and safety limits of the MRI system.

16. The system of claim 1, wherein at least one of the one or more LLMs was trained on patient data paired with manually generated imaging protocols.

17. The system of claim 1, wherein at least one of the one or more LLMs was trained on patient data paired with imaging protocols and recall rates associated with the imaging protocols.

18. A method for automating generation of a magnetic resonance imaging protocol for a patient, the method comprising using one or more processors to perform steps of:
receiving information comprising patient data;
accessing one or more trained LLMs;
applying the patient data to the one or more trained LLMs to generate an MRI protocol comprising one or more pulse sequences with pulse sequence parameters; and
storing the MRI protocol.

19. The method of claim 18, wherein the patient data comprises at least one of an imaging order posing a clinical question or a patient identification.

20. The method of claim 18, wherein the steps further comprise:
generating an event table of the one or more pulse sequences, the event table comprising at least one of radiofrequency (RF) pulse blocks, gradient waveform blocks, analog to digital converter blocks, or timing parameters;
communicating the event table to an MRI system and controlling the MRI system to acquire imaging data based on the event table;
applying the imaging data to one of the one or more trained LLMs to generate a reconstruction pipeline; and
applying the reconstruction pipeline to the imaging data to generate images.

21. The method of claim 18, further comprising training one of the one or more LLMs by accessing training data and training the LLM using the training data as input data to generate an MRI protocol, wherein the training data comprises MRI protocols paired with at least one of medical records of a plurality of patients or image orders that pose clinical questions generated for the plurality of patients, each comprising a pulse sequence and pulse sequence parameters.

22. The method of: claim 21, wherein the training data further comprises recall rates paired with the MRI protocols.

23. A system for automating an MRI pipeline for a patient, the system comprising:
one or more processors configured to:
receive information comprising patient data;
access one or more trained LLMs;
apply the patient data to the one or more trained LLMs to generate an MRI protocol comprising one or more pulse sequences with pulse sequence parameters; and
store the MRI protocol.
